(19) United States
(12) Patent Application Publication
(10) Pub. No.: US 2024/0278345 A1
(43) Pub. Date: Aug. 22, 2024

(54) METHOD OF MANUFACTURING ONE OR MORE SHARP BODIES BY WIRE ELECTRO-EROSION, SEMIFINISHED PRODUCT, FIXTURE, AND METHOD OF MANUFACTURING A SURGICAL CUTTING INSTRUMENT FOR ROBOTIC MICROSURGERY BY WIRE ELECTRO-EROSION

(71) Applicant: MEDICAL MICROINSTRUMENTS, INC., Wilmington, DE (US)

(72) Inventors: Neri PIEROTTI, Pisa (IT); Giorgio LAZZARI, Pisa (IT); Marco BACCHERETI, Pisa (IT); Massimiliano SIMI, Pisa (IT)

(21) Appl. No.: 18/570,548

(22) PCT Filed: Jun. 16, 2022
(86) PCT No.: PCT/IB2022/055600

§ 371 Date: Dec. 14, 2023

(30) Foreign Application Priority Data
Jun. 21, 2021 (IT) 102021000016163

(51) Int. Cl. B23H 9/08; A61B 17/00; A61B 17/3211; B23H 7/02; B23H 7/26; B23H 11/00

(52) U.S. Cl. CPC B23H 9/08; A61B 17/3211; B23H 7/02; B23H 7/26; B23H 11/003; A61B 2017/00526; B23H 2600/10

(57) ABSTRACT

A method of manufacturing sharp bodies by wire electro-erosion includes providing a wire electro-erosion machine and a fixture mounted to the wire electro-erosion machine so that a portion thereof can rotate about a rotation axis which is transverse to a longitudinal extension of the cutting wire. At least one workpiece is mounted to the fixture. An edge to be sharpened of the at least one workpiece is sharpened by a sharpening through cut with the cutting wire on the at least one workpiece. The at least one workpiece is shaped by a shaping through cut with the cutting wire on the at least one workpiece. Between the sharpening and shaping, the portion of the fixture is rotated about the rotation axis thereof by a sharpening rotation angle other than 90°. The sharp bodies may be blades for a surgical cutting instrument for robotic microsurgery.

### FIELD OF THE INVENTION

The present invention relates to a manufacturing method by wire electro-erosion.

In particular, the method according to the present invention is adapted to make one or more sharp bodies.

Furthermore, the present invention relates to a manufacturing fixture.

The one or more sharp bodies made with the method according to the invention are particularly adapted to be miniaturized cutting components.

The one or more sharp bodies made with the method according to the invention are particularly suitable, but not uniquely intended, for a surgical cutting instrument.

Furthermore, the present invention relates to a surgical cutting instrument having one or more sharp bodies made according to the method.

The present invention further relates to a semi-finished product.

Furthermore, the present invention relates to a method of manufacturing an articulated end-effector of a surgical cutting instrument by wire electro-erosion.

### Background Art

Robotic surgery apparatuses are generally known in the art and typically comprise a central robotic tower (or cart) and one or more robotic arms extending from the central robotic tower. Each arm comprises a motorized positioning system (or manipulator) for moving a surgical instrument distally attachable thereto, in order to perform surgical procedures on a patient. The patient typically lies on an operating bed located in the operating room, in which sterility is ensured to avoid bacterial contamination due to non-sterile parts of the robotic apparatus.

Surgical cutting instruments are generally known which are typically provided with a pair of blades to perform a cutting action, such as surgical instruments of the surgical scissor type or of the needle-driver/suture-cutter type. The blades are typically made by molding or deep drawing and then are sharpened by grinding. These known techniques for molding and grinding blades for surgical cutting instruments impose limitations on the miniaturization of the active part of the surgical cutting instrument and are also linked to the support capacity of the piece during the sharpening action by grinding, as well as to the resistance and maintenance of the shape of the piece itself subject to significant external forces from the sharpening process

U.S. Ser. No. 10/864,051, WO-2017-064301, WO-2019-220407, WO-2019-220408, WO-2019-220409 and US-2021-059776 to the same Applicant disclose teleoperated robotic surgery systems having one or more surgical instruments controlled by one or more master interfaces.

Furthermore, documents U.S. Ser. No. 10/582,975, EP-3586780, WO-2017-064303, WO-2017-064306, WO-2018-189721, WO-2018-189722, WO-2018-189729, US-2020-0170727 and US-2020-0170726 to the same Applicant disclose various embodiments of surgical instruments suitable for robotic surgery and microsurgery. These types of surgical instruments typically comprise a proximal interface transmission portion (or backend portion) having an interface intended to be driven by a robotic manipulator, a shaft, and an articulated cuff at the distal end of the shaft. The articulated cuff consists of a plurality of links moved by a plurality of tendons (or actuation cables). One or more terminal links have a free end and are adapted to operate directly on a patient's anatomy and/or handle a needle as well as a suture thread for performing anastomoses or other surgical therapies.

Furthermore, WO-2017-064305, EP-3362218 and EP-3597340 to the same Applicant disclose methodologies for manufacturing a surgical instrument including wire electro-erosion, also known with the terminology “WEDM”, “wire-cut”, “electro-erosion”, “spark-machining”, or “spark-eroding”.

In addition, document FR-2867995 shows a wire electro-erosion process for making optical components which provides workpieces capable of rotating about the longitudinal axis thereof.

Therefore, the need is felt to provide a manufacturing process capable of making one or more miniaturized sharp bodies.

Therefore, the need is felt to provide a manufacturing process capable of making one or more miniaturized sharp bodies ensuring high precision in creation and repeatability.

Therefore, the need is felt to provide a manufacturing process capable of making one or more miniaturized sharp bodies having one or more sharp sides and one or more shaped sides with the same manufacturing process.

Therefore, the need is felt to provide a single manufacturing process capable of carrying out both the sharpening step and the shaping step.

In particular, in the medical-surgical field, the need is felt to provide a manufacturing process solution capable of making one or more miniaturized blades for making a miniaturized surgical cutting tool.

In particular, the need is felt to provide a robust, repeatable and serializable manufacturing process capable of producing one or more miniaturized blades in an economically sustainable manner for single-use surgical instrumentation.

### Solution

It is an object of the present invention to solve the described drawbacks of the prior art and provide a solution to the needs mentioned above.

This and other objects are achieved with a method according to claim 1, as well as with a semi-finished product according to claim 20, as well as with a fixture according to claim 21.

Some advantageous embodiments are the subject of the dependent claims.

According to an aspect of the invention, a method for making one or more sharp bodies by wire electro-erosion comprises the steps of: (i) providing a wire electro-erosion machine having a cutting wire and providing a fixture mounted to the wire electro-erosion machine, in which the fixture is mounted so that at least one portion thereof can rotate about a rotation axis which is transverse to the longitudinal extension of the cutting wire; (ii) mounting at least one workpiece to the fixture; (iii) sharpening at least one edge to be sharpened of the at least one workpiece by performing with the cutting wire a sharpening through cut on the at least one workpiece; (iv) shaping the at least one workpiece by performing with the cutting wire of a shaping through cut on the at least one workpiece.

According to an aspect of the invention, between the sharpening step and the shaping step, the further step is performed of rotating the at least one portion of the fixture about the rotation axis thereof by a sharpening rotation angle other than 90°. Such a sharpening rotation angle can be identical to the angle formed in the cross-section of the cutting edge made on the workpiece.

The sharpening rotation angle can be chosen to minimize the movement of the workpiece with respect to a cutting wire head of the electro-erosion machine.

The sharpening rotation angle can be an acute angle.

By virtue of such a method, replacements of the workpiece on the fixture are avoided.

The one or more sharp bodies can comprise one or more surgical blades.

The method can make a plurality of sharp bodies on the same workpiece in which the sharpening and shaping steps are the same for all the sharp bodies of said plurality. The sharpening step can be carried out by a single cutting trajectory (or a single cutting path) having a start point and an end point which determines the sharpening of a plurality of edges to be sharpened. The shaping step can be carried out by a single cutting trajectory (or a single cutting path) having a start point and an end point which determines the shaping of a plurality of pieces to be machined.

The shaping step can comprise the step of separating the sharp bodies. A step can be included of collecting the separate sharp bodies in a collection basket by gravity. Therefore, the collection basket can be arranged below, i.e., lower with respect to the cutting wire.

The sharpening step can be carried out before the shaping step. The shaping through cut can cross at least one portion of the sharp edge during the sharpening step. For example, the cutting path of the shaping through cut can be directed locally transversely to the sharp edge and cross it, resulting in a shaping of the sharp edge.

The workpiece can comprise a plate-like body, such as a plate, a strip, a belt, and the sharpening and shaping steps each include making a through cut through the thickness of the plate-like body of the workpiece. The thickness of the plate-like body can be less than 1 millimeter, such as between 0.05 and 0.5 millimeters. The plate-like body can be an elastic body which can be elastically deformable by bending, for example made of steel for blades.

The sharp edge can be a curved edge in a definable lying plane of the sharp body.

The shaping step can comprise making at least one hole edge intended to delimit a through hole through the thickness of the sharp body, for example said through hole can be a centering hole, in which the hole edge can have an open profile defining a cutting channel on the body of the piece due to the passage of the cutting wire.

The mounting step can comprise assembling to the fixture a plurality of workpieces, in which the sharpening and shaping steps are performed by individually sharpening and shaping each workpiece of said plurality.

The fixture can be made so that the individual pieces to be machined can be machined individually by the cutting wire on at least two cutting planes misaligned from each other by said sharpening rotation angle. In other words, the workpieces to be machined can be mounted to the fixture so that the cutting edge, which extends substantially straight, intersects at most one of the workpieces to be machined at a time on each cutting plane provided.

The fixture can include fixing multiple planar elements (strips) which are individually machinable by wire electro-erosion in one or more rotation configurations about the rotation axis.

After the shaping step, a step of reshaping the workpiece can be included on a second, different cutting plane by performing a second shaping through cut on the workpiece, in which between the shaping step and the reshaping step the fixture has completed a rotation which can be substantially equal to 90°. The sharpening step can be carried out between the shaping step and the reshaping step. The reshaping step can be performed on a sub-group of workpieces.

A zeroing and calibration strategy of the electro-erosion machine can be included, which includes identifying a point of origin by contacting a known reference on the fixture and/or workpiece with the cutting wire. According to an implementation, the method comprises the further steps of identifying a point of origin or reference of the cutting path and approaching, for example until reaching, the point of origin or reference with the cutting wire. The point of origin can belong to the workpiece, such as the edge of the workpiece to be sharpened.

The point of origin or reference can be a single point of origin for both the sharpening step and the shaping step, as well as for the reshaping step, and the control system of the wire electro-erosion machine can store said single point of origin or reference and relate it geometrically (e.g., trigonometrically) to the kinematic rotation of the fixture of said sharpening rotation angle to process the next cutting path. The sharpening cut and the shaping cut can both start from the same point which is in geometric relation to the point of origin or reference. After the identification step and before the sharpening and/or shaping step, it is possible to perform a rotation of the fixture about the rotation axis by a certain angle which can be an acute angle.

The sharpening through cut can be performed with repeated multiple passes of the cutting wire along a same sharpening cutting path, and the number of said repeated multiple passes of the cutting wire to perform said sharpening through cut is greater than the number of passes made to perform the shaping through cut.

The sharpening of the sharp edge carried out can be a “no back bevel” or “chisel edge” type sharpening, according to a terminology known in the field.

The shaping step can include not separating the sharp bodies and leaving at least one bridge of material for each sharp body intact.

According to an aspect of the invention, a semi-finished product is provided comprising a plate-like body, for example a sheet-like body, having in a single piece a plurality of sharp bodies shaped and connected together by connecting bridges.

According to an aspect of the invention, a fixture is provided for an electro-erosion machine having a fixing portion to the machine and a housing portion for receiving at least one workpiece, in which the housing portion is rotatable with respect to the fixing portion. A motor can be provided for performing the rotation.

The fixture can comprise a plurality of seats for receiving workpieces.

According to an aspect of the invention, a method for manufacturing a articulated surgical cutting instrument by wire electro-erosion comprises the following steps of: (i) providing a wire electro-erosion machine comprising a cutting wire and a fixture which is rotatable with respect to the cutting wire about a rotation axis which is transverse to the longitudinal extension of the cutting wire; (ii) assembling a plurality of workpieces to be machined on the fixture; (iii) sharpening at least one edge to be sharpened of at least one workpiece of said plurality by performing with the cutting wire a sharpening through cut on the at least one workpiece; (iv) shaping on a first cutting plane at least some of, but also all, the workpieces of said plurality one at a time; (v) reshaping on a second cutting plane at least some, but also all, of the workpieces of said plurality by performing a shaping through cut with the cutting wire on said at least some of, but also all, the workpieces of said plurality one at a time, in succession.

According to an aspect of the invention, between the sharpening step and the shaping step on a first cutting plane, the step of rotating the fixture by a sharpening rotation angle different from 90° is performed.

In other words, the sharpening step and the shaping step on a first cutting plane, the fixture has completed a rotation of a sharpening angle other than 90°.

According to an aspect of the invention, between the shaping step on a first cutting plane and the reshaping step on a second cutting plane, the step of rotating the fixture about the rotation axis thereof by a rotation angle preferably substantially equal to 90° is provided.

At least one of the workpieces of said plurality can be a small cylinder of material.

The arrangement of the workpieces of said plurality of workpieces on the jig preferably must meet the condition that the cutting wire intersects at most one of the workpieces at a time in each cutting step (i.e., sharpening, shaping and reshaping).

The method can comprise the step of separating the shaped pieces.

The method can comprise the step of assembling the separate pieces together, in which at least one of the pieces has a sharp edge.

According to an aspect of the invention, a collection basket is provided for collecting the sharp, shaped and separate bodies, in which the collection basket is mounted on an electro-erosion machine.

### BRIEF DESCRIPTION OF THE DRAWINGS

Further features and advantages of the invention will appear from the following description of preferred embodiments, given as an indication and not as a limitation, with reference to the accompanying drawings (it should be noted that references to “an” embodiment as well as to “an” operating mode in this disclosure do not necessarily refer to the same embodiment or operating mode, and are to be understood as at least one, furthermore, for the purposes of conciseness and reduction of the total number of drawings, a certain drawing can be used to show the features of more than one embodiment as well as more than one operating mode, and not all elements of the drawing may be necessary for a certain embodiment/operating mode), in which:

FIG. 1-A is a block diagram showing some possible steps of a method according to a possible operating mode;

FIGS. 1-B and 1-C are block diagrams showing some possible steps of a method according to some possible operating modes;

FIG. 2 is a block diagram showing some possible steps of a method according to a possible operating mode;

FIG. 3 is a diagram in vertical elevation showing a wire electro-erosion machine, according to an embodiment;

FIG. 4-A is a plan view showing a portion of the wire electro-erosion machine of FIG. 3;

FIG. 4-B shows a vertical elevation view of a jig, according to an embodiment;

FIG. 4-C shows an axonometric view of a rotatable portion of the jig of FIG. 4-B;

FIG. 5-A shows an axonometric view of a sharpening step, according to a possible operating mode;

FIG. 5-B shows a vertical elevation view of a jig assembling a workpiece at the end of a sharpening step, according to a possible operating mode;

FIG. 5-C is a cross-section diagram of a workpiece diagrammatically showing a sharpening step, according to a possible operating mode;

FIG. 5-D is a cross-sectional diagram of a workpiece at the end of a sharpening step, according to an embodiment;

FIG. 5-E is a cross-section diagram of a workpiece diagrammatically showing a sharpening step, according to a possible operating mode:

FIG. 5-F is a cross-sectional diagram of a workpiece at the end of a sharpening step, according to an embodiment;

FIG. 6-A shows an axonometric view of a rotation step, according to a possible operating mode;

FIG. 6-B shows a vertical elevation view of a rotation step, according to a possible operating mode;

FIG. 7-A shows an axonometric view of a shaping step, according to a possible operating mode;

FIG. 7-B is an enlargement of the circled detail of FIG. 7-A;

FIG. 7-C shows in cross-section a workpiece subjected to sharpening and shaping, according to a possible operating mode;

FIG. 8-A shows a blade in vertical elevation, according to an embodiment;

FIG. 8-B diagrammatically shows a step of curving, according to a possible operating mode;

FIG. 8-C shows a blade in vertical elevation, according to an embodiment;

FIG. 8-D shows a blade in vertical elevation, according to an embodiment;

FIG. 8-E shows a blade in vertical elevation, according to an embodiment;

FIG. 9-A shows a plan view of a sharpening cutting path and a shaping cutting path, in accordance with a possible operating mode;

FIGS. 9-B and 9-C show a plan view of two possible passes of a shaping step, according to a possible operating mode;

FIGS. 10-A and 10-B show a plan view of two possible passes of a shaping step, according to a possible operating mode;

FIG. 11 shows a plan view of a shaping step, according to a possible operating mode;

FIG. 12 shows a plan view of a shaping step, according to a possible operating mode;

FIG. 13-A shows a semi-finished product, according to an embodiment, obtainable from the shaping step shown in FIG. 11;

FIG. 13-B shows a semi-finished product, according to an embodiment, obtainable from the shaping step shown in FIG. 12;

FIG. 14 is an electron microscope image showing two sharp bodies placed on top of a 5 cent euro coin, according to some embodiments;

FIG. 15 is an electron microscope image showing a blade in vertical elevation, according to an embodiment;

FIG. 16 is a photographic image showing a collection basket for a wire electro-erosion machine, according to an embodiment;

FIG. 17 shows an axonometric view of a surgical instrument, according to an embodiment;

FIG. 18-A shows an axonometric view with separate parts of a portion of an end-effector of a surgical instrument, according to an embodiment;

FIGS. 18-B, 18-C and 18-D show a plan view with separate parts, a plan view with assembled parts, and an axonometric view with separate parts, respectively, of a portion of the end-effector of FIG. 18-A;

FIG. 19 shows an axonometric view with separate parts of a portion of an end-effector of a surgical instrument, according to an embodiment;

FIG. 20-A shows an axonometric view with separate parts of a portion of an end-effector of a surgical instrument, according to an embodiment;

FIGS. 20-B and 20-C show a vertical elevation view and a plan view, respectively, of a portion of the end-effector of FIG. 20-A in a closed configuration;

FIG. 21 shows an axonometric view of a portion of an end-effector of a surgical instrument, according to an embodiment;

FIG. 22 shows an axonometric view with separate parts of a portion of an end-effector of a surgical instrument, according to an embodiment;

FIG. 23 shows an axonometric view of a robotic system for surgery, according to an embodiment;

FIGS. 24-A, 24-B and 24-C show a sequence of sharpening, rotating and shaping steps, according to some possible operating modes;

FIGS. 25-A, 25-B and 25-C show a sequence of sharpening, rotating and shaping steps, according to some possible operating modes:

FIGS. 26, 27 and 28 show some possible steps of a method according to some possible operating modes, as well as some embodiments of a fixture;

FIGS. 29-A, 28-B and 29-C show a sequence of sharpening, rotating and shaping steps, according to some possible operating modes;

FIG. 29-D is a diagrammatic view according to the point of view indicated by arrow D of FIG. 29-C;

FIG. 30 shows an axonometric view of a fixture in accordance with an embodiment which assembles a plurality of workpieces;

FIG. 31 diagrammatically shows a vertical elevation view of a possible step of a method, according to a possible operating mode;

FIGS. 32-A, 32-B and 32-C diagrammatically show in vertical elevation some possible steps of a method, according to some possible operating modes.

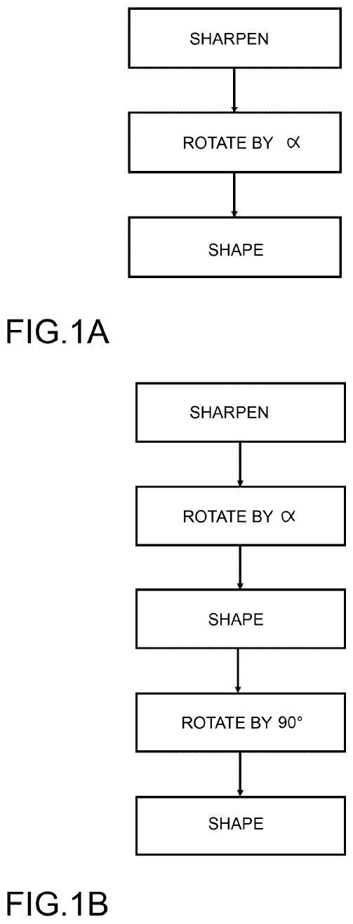

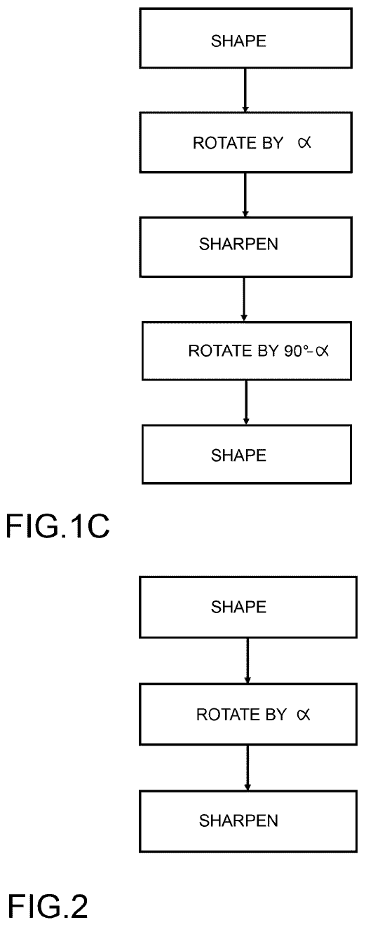

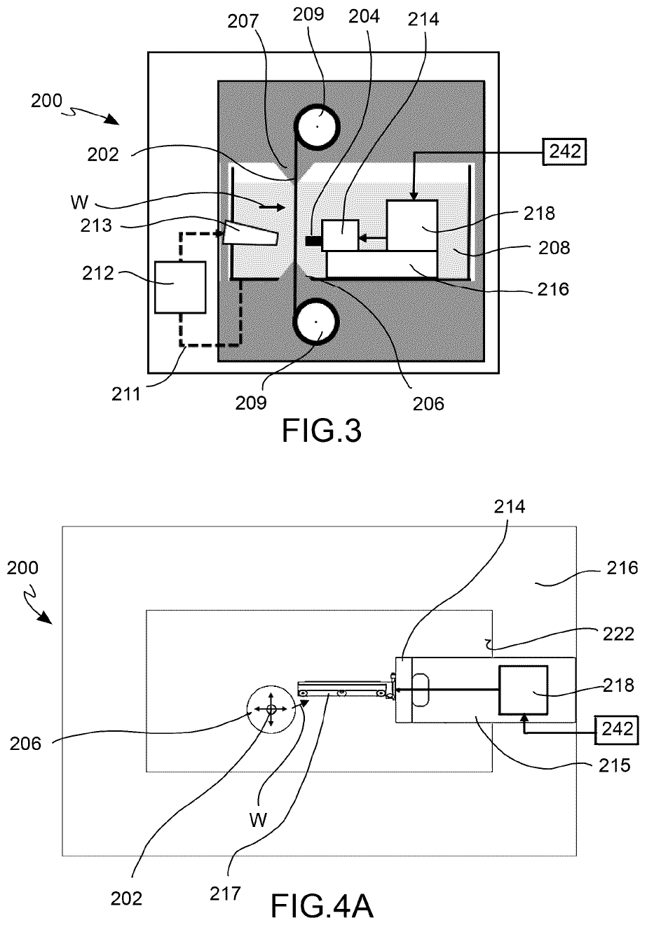

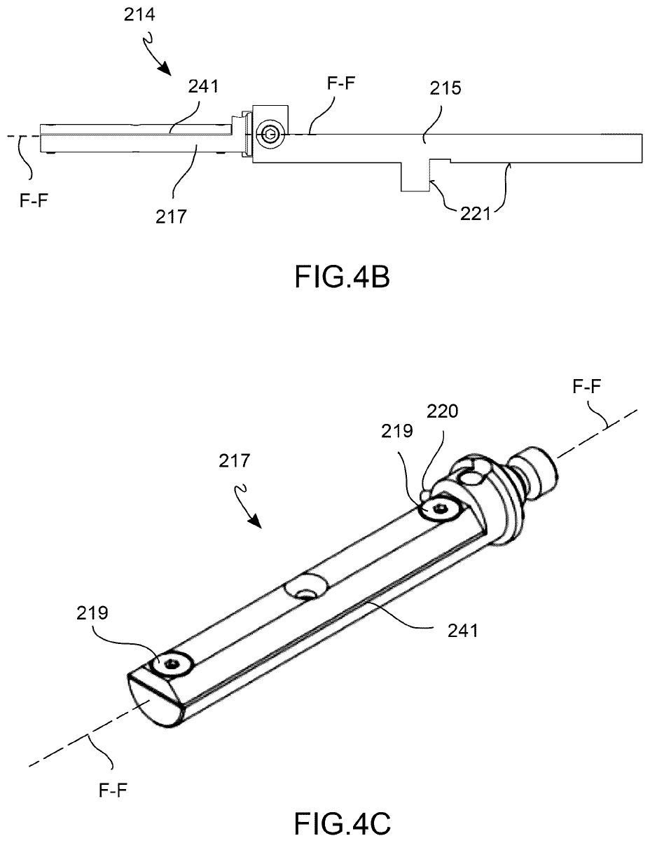

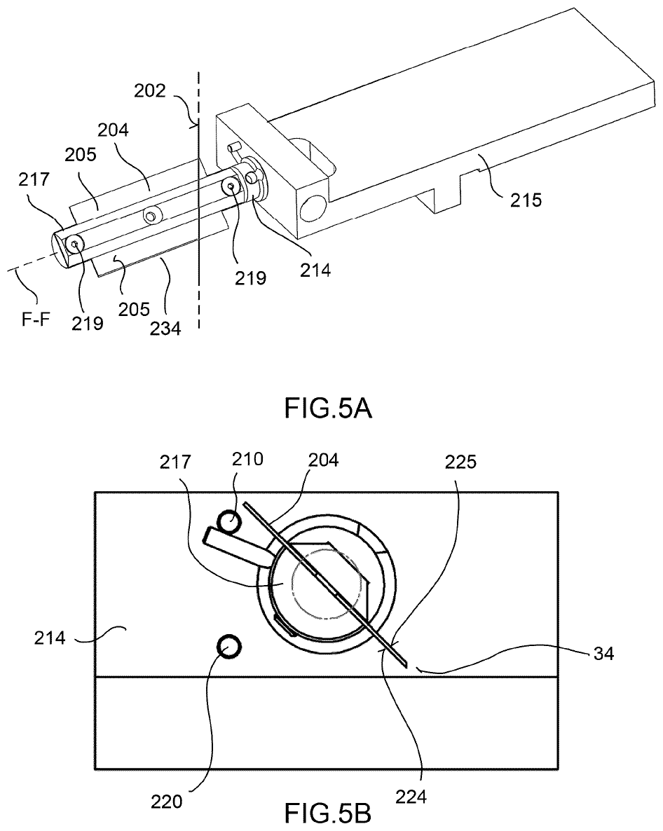

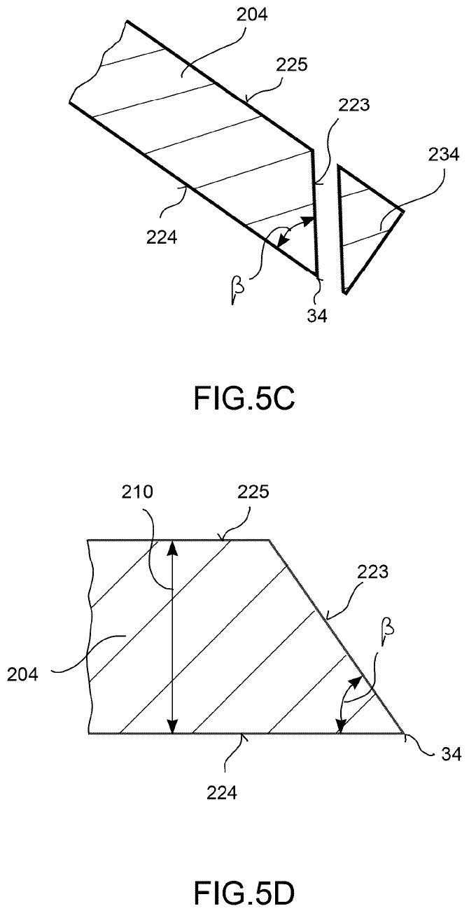

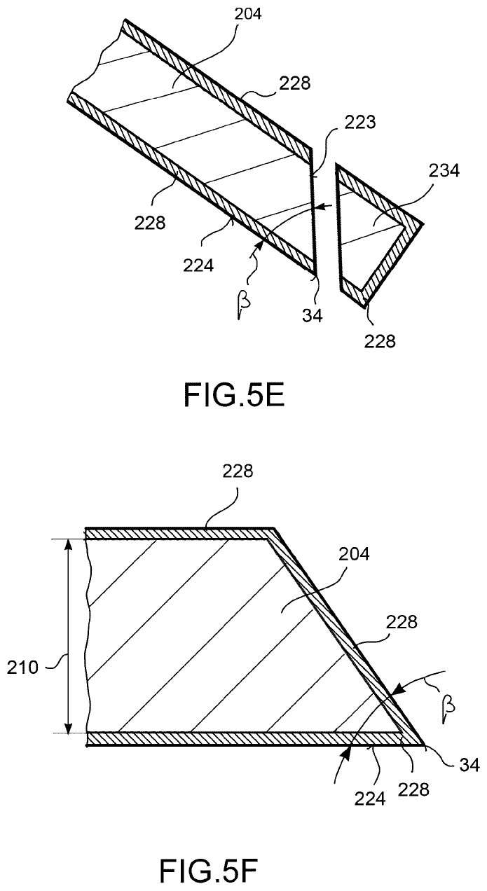

### DETAILED DESCRIPTION OF SOME EMBODIMENTS

Reference throughout this description to “an embodiment” is meant to indicate that a particular feature, structure or function described in relation to the embodiment is included in at least one embodiment of the present invention. Therefore, the formulations “in an embodiment” in various parts of this description do not necessarily all refer to the same embodiment. Furthermore, particular features, structures or functions such as those shown in different drawings can be combined in any suitable manner in one or more embodiments. Similarly, reference throughout this description to “an operating mode” is meant to indicate that a particular feature, structure or function described in connection with the operating mode is included in at least one operating mode of the present invention. Therefore, the formulation “in an operating mode” in various parts of this description does not necessarily all refer to the same operating mode. Furthermore, particular features, structures or functions such as those shown in different drawings can be combined in any suitable manner in one or more operating modes.

In accordance with a general embodiment, a method of manufacturing one or more sharp blades is provided. Such one or more sharp bodies are preferably intended to form miniaturized cutting elements.

In accordance with a preferred operating mode, the manufacturing method is adapted to make one or more blades. Such one or more blades are preferably miniaturized blades.

The method comprises the step of providing a wire electro-erosion machine 200 comprising a cutting wire 202.

The cutting wire 202 preferably extends longitudinally between two heads 206, 207 of the wire electro-erosion machine 200 when in operating conditions. To perform the cut (i.e., electro-erosion), the cutting wire 202 advances along a cutting path in a feeding direction W (or cutting direction W) which is substantially orthogonal to the longitudinal extension of the cutting wire 202, i.e., the feed direction is substantially orthogonal to the sliding direction of the portion of the cutting wire 202 between the two heads 206, 207 of the machine 200, in a manner known per se. Each of the two heads 206, 207 can be associated with a reel 209 or winding/unwinding roller 209 for the cutting wire 202. When in operating condition, the cutting wire 202 runs winding on one reel as it unwinds from the other reel, and the heads 206, 207 guide the cutting wire 202 in the feeding direction W (or cutting direction W) to perform a cut on the workpiece.

The wire electro-erosion machine 200 preferably comprises a tank 208 to be filled with dielectric liquid inside which the electro-erosion of at least one workpiece 204 occurs when in operating conditions. The electro-erosion machine 200 can further comprise a hydraulic circuit comprising a hydraulic duct 211 fitted with a pump 212 and a filter which withdraws and filters dielectric fluid from the tank 208 and ending with a nozzle 213 which directs dielectric fluid onto the workpiece 204.

The at least one workpiece 204 is preferably made of electrically conductive material, such as metal, or is coated with electrically conductive material.

The wire electro-erosion machine 200 further comprises at least one jig 214 or fixture 214 which is rotatable with respect to the cutting wire 202 (i.e., with respect to the cutting section of the cutting wire 202) about a rotation axis F-F which is transverse, and preferably orthogonal, to the longitudinal extension of the cutting wire 202. For example, the rotation axis F-F of the jig 214 extends substantially horizontally while the cutting portion of the cutting wire 202 substantially vertically.

The method comprises the step of mounting at least one workpiece 204 on the jig 214, for example by fixing the workpiece 204 by fixing screws or other fasteners to the jig 214 such that the at least one workpiece 204 is integral in rotation with a portion of the jig 214. Thereby, rotating the jig 214 about the rotation axis F-F thereof results in a rotation of the workpiece 204 with respect to the cutting wire 202.

The jig 214 can comprise a fixing portion 215 fixed to a bracket of the worktop 216 inside the tank 208 of the wire electro-erosion machine 200, and a housing portion 217 receiving said at least one workpiece 204 for example in at least one of the housing seats 241 thereof, in which the housing portion 217 of the jig 217 is rotatable with respect to the fixing portion 216 to the machine 200 about said rotation axis F-F. In accordance with an embodiment, the fixing portion 216 to the machine 200 of the jig 214 comprises positioning rectified surfaces 221 intended to abut against rectified counter-surfaces 222 of the bracket of the worktop 216 of the machine 200.

The housing portion 217 of the jig 214 can have an elongated body extending along the rotation axis F-F and can be pivotally connected to the fixing portion 215. Rotating only the housing portion 217 with respect to the fixing portion 215 allows minimizing the translation movements of the workpiece 204 with respect to the lower head 206 of the machine which can derive from the rotation step, as it is generally desirable to position the workpiece 204 close to the lower head 206 during cutting to minimize the deformability of the cutting wire 202. In other words, a rotation of the jig could move the workpiece with respect to the cutting wire in the longitudinal extension direction of the cutting wire between the machine heads, for example bringing the workpiece located close to a head located at the median zone of the section of the cutting wire extended between the heads of the machine, which is more deformable transversely with respect to the section close to one of the heads with consequent variation of the cutting features, for example in terms of finish and/or cutting resolution. Typically in fact, a wire electro-erosion machine is adapted to perform a better and more precise cutting machining when the workpiece is arranged close to at least one of the heads where the cutting wire is less transversely deformable while sliding longitudinally, as well as when the heads are close to one another shortening the longitudinal extension of the portion of the cutting wire extending between the machine heads to limit the transverse movements thereof when in operating conditions, i.e., cutting, as well as when the sliding direction of the wire is perfectly orthogonal to the plane identified by the feeding direction W or cutting direction W. The electro-erosion machine 200 can be provided with the functionality which includes crossing the heads 206, 207, i.e., translating the heads so as to incline the cutting wire 202 with respect to the workpiece 204, but in light of the above to obtain a satisfactory cutting accuracy the heads must be kept close and therefore such a functionality of crossing the heads allows to incline the cutting wire with respect to the workpiece at a maximum of an angle around 5°, in general terms, which makes this solution of crossing the heads of the wire electro-erosion machine unsuitable for obtaining a sharpening.

The housing seat 241 of the housing portion 217 of the jig 216 can be formed by a longitudinal slot 241 along the body of the housing portion 217 for receiving a workpiece 204 which is a plate-like body, tightening it, for example by clamping and positioning elements 219, in a central portion thereof so that the plate-like body of the workpiece 204 forms two opposite cantilevered flaps 205 which can both be subject to wire electro-erosion machining. The workpiece 204 can be tightened in other manners. Positioning elements such as holes or notches can be provided on the body of the workpiece for mounting the workpiece to the jig 214.

Preferably, the extension of the cantilevered portion of each cantilevered flap 205 of the plate-like body of the workpiece 204 projecting cantilevered from the housing portion 217 of the jig 214 is chosen so as to minimize the vibrations which can arise during the action of the cutting wire 202 on the workpiece 204 as well as on the jig 214 and which would lead to cutting uncertainty. Screws or tightening screws can be provided as tightening and positioning elements 219 adapted to tighten the housing seat and meanwhile acting as positioning elements of the workpiece 204 in the seat. In accordance with a possible operating mode, one or more fixing and positioning elements 219 are designed to cross the body of the workpiece 204 for example in a through hole thereof in order to exert the fixing action thereof to the jig and positioning action with respect to the jig and the cutting edge.

In accordance with a possible operating mode, the workpiece 204 comprises a plate-like body having a thickness 210 in the range from 0.05 mm to 0.5 mm. The plate-like body can be obtained from a strip tape of material or from a full piece of sliced material. The plate-like body can be a deformable elastic body in bending.

The method comprises the step of sharpening at least one edge to be sharpened 234 of the at least one workpiece 204 by making at least one sharpening through cut with the cutting wire 202 on the at least one workpiece 204. The advancement of the cutting wire 202 along a sharpening cutting path makes a through cut on the at least one workpiece which results in the sharpening of at least one edge to be sharpened 234 of the workpiece 204 making the edge to be sharpened 234 a sharp edge 34.

The at least one sharp edge 34 will form the cutting edge of the sharp body manufactured with the method. Therefore, the at least one sharp edge 34 will form the cutting edge of the body of the one or more blades 30 manufactured with the method, where the manufacturing method is employed to make one or more blades.

The method further comprises the step of shaping the at least one workpiece 204 by performing at least one shaping through cut on the at least one workpiece 204 with the cutting wire 202. The advancement of the cutting wire 202 along a shaping cutting path 230 makes a through cut on the at least one workpiece 204 which results in the shaping of the sharp body, for example of the one or more blades 30 made with the manufacturing method. Not necessarily, the shaping step results in the separation of the single sharp body and for example a bridge 231 of material can connect the sharp bodies together at the end of the shaping step. The shaping step can make an end 32 on the workpiece which can form the distal end of the sharp body.

Of course, the sharpening and shaping steps can be performed in any order.

Advantageously, between the sharpening step and the shaping step, the further step of rotating the jig 214 about the rotation axis F-F thereof by a sharpening rotation angle α is performed.

In accordance with an embodiment, a motor 218, for example an electric motor, is associated with the jig 214 to rotate the housing portion 217 of the jig 214 with respect to the fixing portion 215. In such a case, the step of rotating the jig 214 is performed by operating the motor 218. The electro-erosion machine 200 also preferably comprises at least one electronic control system 242 and the motor 218 is operatively connected to said electronic control system 242 of the machine 200. Therefore, it is possible to automate the step of rotating the jig 214.

With further advantage, the sharpening rotation angle α is different from 90°.

“Different from than 90°” is meant to indicate an angle significantly different from 90°, in which the deviation from 90° is at least 10°, i.e., the sharpening rotation angle α is different from 90°±10°. Preferably, it is meant to indicate a sharpening rotation angle α different from 90° in absolute value, i.e., in any rotation direction (clockwise or counterclockwise) about the rotation axis F-F.

The provision of a sharpening angle α other than 90° allows to make an acute angle β in the cross-section of the workpiece body, forming a sharp edge 34.

In accordance with a preferred embodiment, the sharpening angle α is an acute angle and net of the tolerance of ±10° can be understood as an angle less than 80° in absolute value and preferably greater than 10°.

The sharpening angle α, which measures the rotation of the workpiece with respect to the cutting wire 202, can be chosen so as to achieve the desired cutting performance of the sharp edge 34 because the choice of the sharpening angle α determines the acute angle β in the cross-section of the sharp edge 34.

By virtue of such a method, it is possible to obtain at least two through cuts on the workpiece on two cutting planes which are not orthogonal to each other, in which at least one through cut is sharpening, i.e., it makes a sharp edge 34 on the workpiece 204 and the other through cut is shaping.

Where the workpiece has a plate-like body, preferably the shaping through cut is performed by orienting the cutting wire 202 substantially orthogonally with respect to the plane of the plate-like body, to make cutting walls in the thickness of the short and robust workpiece, while the shaping through cut is performed by orienting the cutting edge obliquely with respect to the plane of the plate-like body, making a sharp profile in the thickness, i.e., in the cross-section, of an edge of the workpiece.

The jig 214 can comprise mechanical stroke ends 220, for example two opposite stroke end ridges 220 facing opposite end stroke abutment surfaces, which are located on the housing portion 217 and on the fixing portion 215 of the jig 214. In such a case, the rotating step can comprise bringing the housing portion 217 of the jig 214 in abutment against a stroke end ridge 220 of the fixing portion 215 of the jig 214. The stroke ends 220 may be releasably associated with the jig 214 so as to allow the sharpening rotation angle α to be adjusted, and for example one or more stroke ends can be extractable and retractable.

The rotating step is performed, avoiding disassembling the workpiece 204 from the jig 214 as well as avoiding disassembling the jig 214 from the wire electro-erosion machine 200. Therefore, replacements are avoided. The rotation axis F-F of the jig 214 can extend through the body of the workpiece 204, for example it can extend along the thickness 210 of the workpiece 204 where the workpiece has a plate-like body (for example it is a strip, a ribbon, a plate, a sheet) and in such a case the rotation of the jig 214 can also result in a rotation of the plate-like body of the workpiece 204 about one of the axes thereof (for example: median axis, axis of symmetry).

By virtue of such a method it is possible to manufacture one or more blades 30 by making two through cuts on the workpiece 204 by wire electro-erosion on two cutting planes which are non-orthogonal to each other and rotated by said sharpening angle α, a through cut being sharpening, and meanwhile it is avoided to disassemble the workpiece 204 from the jig 214 as well as to disassemble the jig 214 from the wire electro-erosion machine 200. Thereby, a high cutting accuracy of the sharpening and shaping cuts is achieved because the at least one workpiece is prevented from being repositioned with respect to the machine, and for example also the calibration of the electronic control system of the electro-erosion machine 200 is more reliable and can be performed only once, for example after the assembly step and before both the sharpening and shaping steps.

To perform the zeroing and calibration of the electro-erosion machine 200, the method can comprise the steps of: identifying reference point 229 and approaching said reference point 229 with the cutting wire 202, prior to the sharpening step. The reference point 229 can be identified by contacting one or more points of the workpiece 204 one or more times with the cutting wire 202. For example, two orthogonal sides of the plate-like body of the workpiece can be contacted to identify a reference point 229 which coincides with a vertex of the plate-like body of the workpiece 204.

In accordance with an operating mode, said reference point 229 belongs to the edge to be sharpened 234 of the workpiece 204.

Not necessarily, the approaching step causes the cutting wire 202 to reach the reference point 229. The cutting start point 232, 235 of the sharpening 240 and/or shaping 230 cutting path can be close to the reference point 229 or coincident with the reference point 229.

In accordance with a possible operating mode, the cutting start point 232, 235 of the sharpening 240 and/or shaping 230 cutting path is placed in a position having a predefined geometric relationship with the reference point 229.

In accordance with a possible operating mode, the identification and approaching steps are performed before each of said sharpening and/or shaping steps.

In accordance with a possible operating mode, the identification and approaching steps come only once, before both the sharpening and shaping steps.

In accordance with a possible operating mode, the identification step comprises identifying a single point of origin of the cutting path which serves as the point of origin for both the sharpening cutting path and the shaping cutting path, and the approaching step comprises approaching said single point of origin with the cutting wire both in preparation for the sharpening step and in preparation for the shaping step.

In accordance with an operating mode, prior to both the sharpening and shaping steps, the method comprises the step of identifying a single point of origin of the cutting path which serves as the point of origin for both the sharpening cutting path and the shaping cutting path, and approaching, preferably until reaching, said single point of origin with the cutting wire 202 both in preparation for the sharpening step and in preparation for the shaping step. Thereby it is possible to reset the machine, i.e., calibrate the machine only once at the beginning of the method, avoiding recalibration.

The identification of said point of origin can be performed by contacting a known reference on said fixture 214 with the cutting wire 202. The identification of said point of origin can be performed by contacting a known reference on said workpiece 204 with the cutting wire 202.

In accordance with a possible operating mode, the method makes a plurality of sharp bodies on a single workpiece 204, and said sharpening step and said shaping step are the same for all the sharp bodies of said plurality. For example, a single sharpening trajectory 240 is provided with starting point 235 and ending point 236 for multiple sharp bodies, whether they are the same or different.

In accordance with a possible operating mode, the sharpening step is performed by a single cutting sharpening trajectory 240 of the cutting wire 202 and said shaping step is performed by a single cutting shaping trajectory 230 of the cutting wire 202. Each cutting trajectory 230, 240 can be subject to multiple repeated passes of the cutting wire.

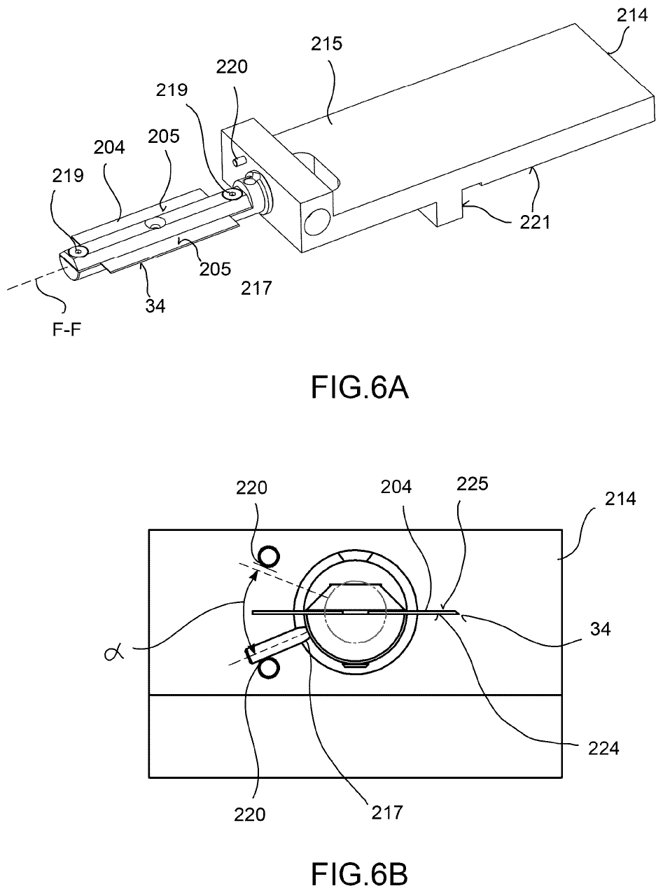

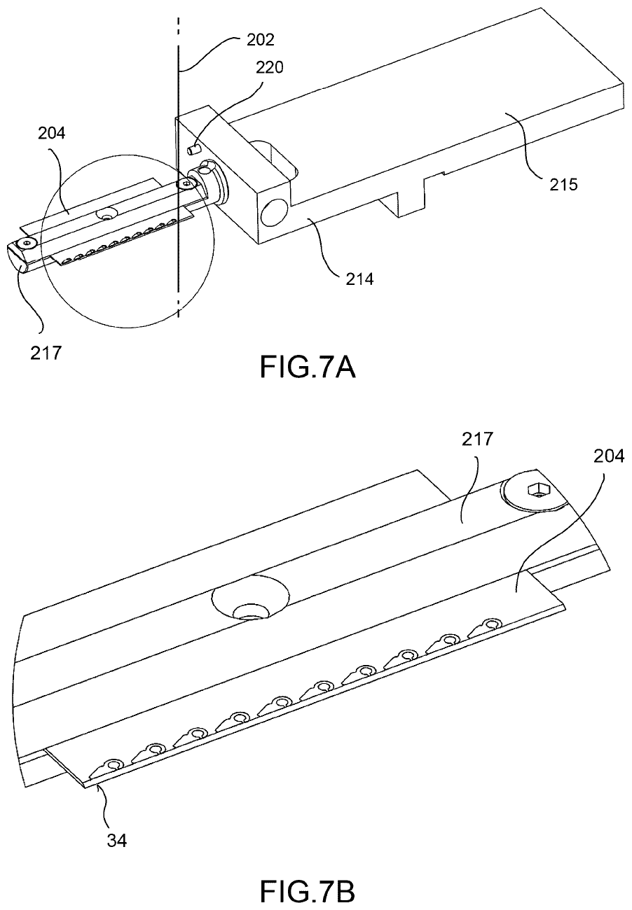

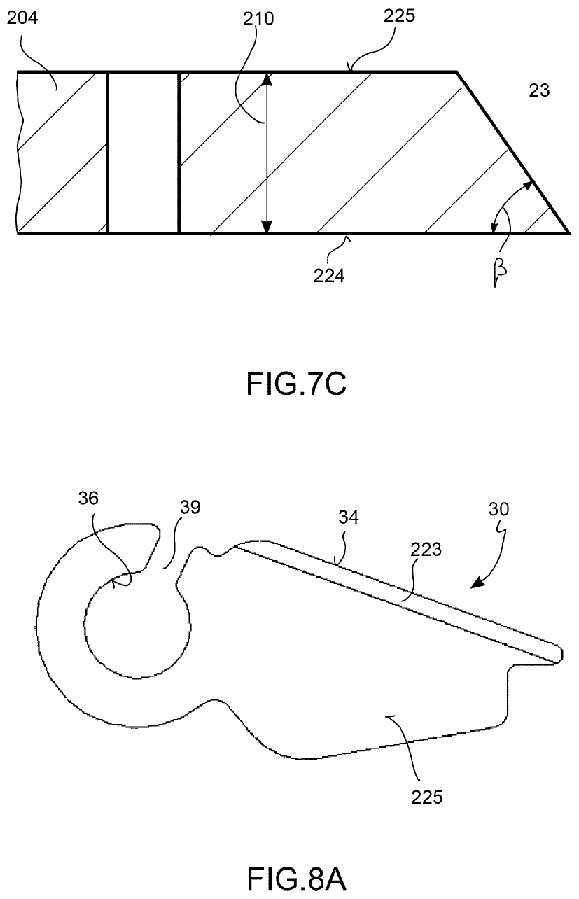

The sharpening through cut removes material from an edge to be sharpened 234 of the workpiece, exposing a sharpening cutting wall 223, in a condition in which the workpiece 204 and the cutting wire 202 form a certain angle with each other (which depends on the choice of the sharpening angle α) chosen so that the exposed sharpening cutting wall 223 and another wall of the workpiece adjacent thereto jointly form a sharp edge 34, i.e., an acute-angled edge defined by the meeting of the sharpening cutting wall 223 and by said other adjacent thereto the workpiece wall. In cross-section, as shown for example in FIG. 6-C, following the sharpening through cut the sharpening cutting wall 223 forms an acute angle β preferably with a face 224 of the back side of the workpiece 204. The sharpening cutting wall 223 can form an acute angle with an opposite face 225, i.e., the front side of the workpiece 204.

Such an acute angle β formed between the sharpening cutting wall 223 and another wall of the workpiece 204 does not necessarily correspond to said sharpening rotation angle α, although in accordance with an operating mode said sharpening rotation angle α is equal to said acute angle β. In accordance with an embodiment, the acute angle β is equal to 90°-α.

In accordance with a possible operating mode in which the workpiece has a plate-like body with parallel opposite faces 224, 225 defining a thickness 210 therebetween, the shaping through cut is performed perpendicularly to the opposite parallel faces 224, 225 through the thickness, and the sharpening through cut is performed in an inclined direction with respect to the opposite parallel faces 224, 225 and across the thickness of the workpiece. Thereby the sharp edge 34 is formed on one face of the opposite parallel faces 224, 225 of the workpiece 204 which is transverse (in this case orthogonal) to the shaping cutting plane and incident to the sharpening cutting plane.

Where the workpiece 204 has a certain geometry, for example but not limited to a planar strip or ribbon or sheet geometry given by the plate-like body thereof, and said sharpening rotation angle α is understood as the rotation angle of the plate-like body during the rotating step, then the acute angle β is in accordance with a preferred embodiment equal to or complementary to the sharpening rotation angle α.

The workpiece 204 can have a squat body or other non-plate-like body and the sharpening through cut is performed through the body of the workpiece 204, forming said sharp edge 34.

The acute angle of the sharp edge 34 must be chosen so as to optimize the cutting performance, finding a compromise between penetration and strength. Typically, an acute angle β of the sharp edge 34 of less than 45°, for example between 10° and 40°, allows for high cutting penetration but tends to wear out early (trend which increases with decreasing acute angle β amplitude) while an acute angle β of the sharp edge 34 greater than 45°, for example between 50° and 80°, allows for long service life but the sharp edge 34 can register resistance to cutting penetration when in service conditions (trend which increases with decreasing acute angle β amplitude).

An acute angle β in the range from 30° to 60° (values to be understood here with a tolerance of ±10%) would offer a satisfactory compromise for applications of the resulting one or more blade(s) 30 in the field of robotic surgery.

In accordance with a preferred embodiment, the acute angle β is substantially equal to 45°. This value can also be understood here with a tolerance of ±10%, although it is preferable here to indicate an acute angle β which is substantially equal to half of 90°, i.e., it makes a through cut exposing a cutting wall significantly facing 45° in the workpiece body.

Accordingly, where the acute angle β depends on the sharpening rotation angle α, said sharpening rotation angle α can be in the range of 20°-70°, and preferably the sharpening rotation angle α is substantially 30°±10° or 45°±10° or 60°±10°. These values are to be understood in absolute value, i.e., they can be valid in any rotation direction of the body of the workpiece 204 with respect to the cutting wire 202 made during the rotation step. Therefore, 45° here means a rotation of 45° in one direction and also an equal rotation of 45° in the opposite rotation direction. The rotation direction has an effect on the direction of the cutting wall 223 exposed on the body of the workpiece 204 and can determine whether the sharp edge 34 belongs to the face of the back side 224 or to the face of the front side 225 of the workpiece 204.

The sharpening angle α can be chosen to minimize the distance between the workpiece and a reference of the machine 200, for example a head 208.

In accordance with a possible operating mode, the sharpening through cut of the sharpening step follows a cutting path 240 extending along the thickness to be sharpened 234 of the workpiece 204. Thereby, it is possible to make a substantially uniform sharp edge 34 along the extension thereof even where the edge to be sharpened 234 has a concave and/or convex geometry in the sharpening cutting plane.

In accordance with a possible operating mode, the edge to be sharpened 234 of the workpiece 204 coincides with a margin of the workpiece body, for example a margin of the plate-like body such as a strip or plate or ribbon and the cutting path 240 of the sharpening through cut extends substantially straight, along the edge of such a margin, and substantially files the edge i.e., electro-erodes material from the thickness 210 of the plate-like body of the workpiece, making a gap which exposes a cutting surface 223 which is inclined with respect to the opposite faces 224, 225 of the plate-like body and forms a sharp edge 34.

Choosing the sharpening rotation angle α can define the direction of the sharpening and shaping through cuts on the workpiece.

In accordance with a preferred operating mode, the shaping through cut crosses the body of the workpiece 204 in the direction of the thickness thereof. In accordance with a preferred operating mode, the shaping through cut produces an edge which is not sharp and for example forms two opposite angles of substantially 90° with the opposite faces 224, 225 of the workpiece, where the workpiece has a predefined regular geometry, for example it is a plate-like body.

The cutting path 230 described by the shaping through cut can form a path comprising curved portions, such as hole edges 36, and in accordance with a possible operating mode making the hole edges 36 involves making radial passage channels 39 for the passage of the cutting wire. The hole edges 36 are not necessarily formed by curved portions and can be formed by broken line segments of hole edges 36. The hole edges 36 can delimit one or more centering holes for receiving an articulation pin when in operating condition.

The curved portions described by the cutting path 230 described by the shaping through cut can make the edge to be sharpened 34 so as to make a curved, concave, and/or convex edge to be sharpened.

The feeding speed parameters of the cutting wire 202 can be adjusted to provide a good compromise between finishing and production times. In accordance with an embodiment, the shaping step makes parts with extreme resolution by means of said through cut, such as legs measuring a few hundredths of a millimeter in width.

In accordance with a possible operating mode, the shaping through cut makes an edge which is not orthogonal with respect to the opposite faces 224, 225 of the workpiece 204, i.e., the shaping cut can make an inclined edge with respect to a definable lying plane of the workpiece.

In accordance with a possible operating mode, first the sharpening step is performed, then the rotating step, then the shaping step. Thereby, the sharpening is done, and the shaping after. In this case, the shaping through cut can cross at least one portion of the sharpening through cut, i.e., the shaping cutting path 230 is incident with the sharpening cutting path. In accordance with this operating mode, the method can allow making a plurality of sharp bodies, for example a plurality of blades 30, from the same workpiece by first sharpening at least one portion of at least one edge of the workpiece 204 which is common to, i.e., shared by, at least one group of bodies, for example blades 30, to be made, and then shaping the individual sharp bodies, for example of the individual blades 30, which includes performing a shaping through cut which crosses the sharp edge 34 and thus cuts the cutting wall 223 to make the individual sharp bodies, for example the individual blades 30, obtainable from the same workpiece 204 separate or separable. For example, where the workpiece is a plate-like body mounted on the jig 214 forming two opposite cantilevered flaps, the method can include first sharpening both said edges and then shaping the individual sharp bodies, for example the individual blades 30, of said plurality on both opposite cantilevered flaps.

In accordance with a possible operating mode, the sharpening step is performed before the shaping step, and in which the shaping cutting path 230 of the shaping step does not extend along the sharp edge 34 made by the sharpening step, i.e., the shaping through cut is not made on the workpiece following the profile of the sharp edge 34 previously machined. The cutting path 230 of the shaping through cut can cross the sharp edge 34 transversely with respect to the longitudinal extension of the edge to shape the blades 30, making an interruption of the sharp edge of the workpiece 204.

In accordance with a possible operating mode, the cutting path 230 of the shaping through cut provides an external section 238 of the cutting path 230 of the workpiece 204 in an external position with respect to the sharp edge 34 and at a certain distance therefrom, in which a calibration verification step is carried out along the external portion 238 of the cutting path 230 which provides a sudden approach of the cutting wire to the sharp edge 34, substantially tracing a notch 239 on the cutting path 230. Thereby, it is possible to verify the correct positioning of the workpiece 204, in fact where the sudden approach of the cutting wire 202 to the sharp edge 34 determines the electro-erosion of material from the sharp edge 202 this would indicate an anomaly, for example a probable positioning error of the workpiece.

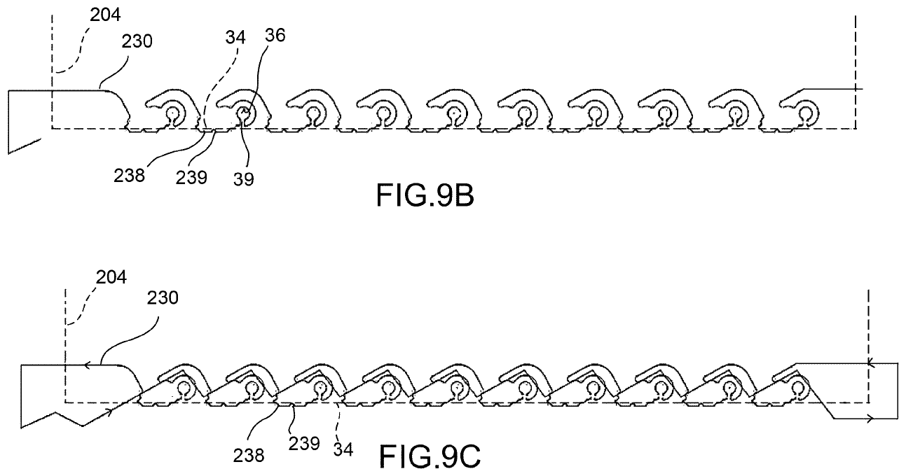

FIG. 9-B shows an example of a shaping cutting path 230 of a shaping through cut which describes the shape of a plurality of blades 30 on the same workpiece, making undercuts, hole edges 36, passage channels 39, said external section 238 with respect to the sharp edge 34. The shaping cutting path 230 shown here can be performed several times, i.e., with multiple repeated passes, for example round trip passes.

FIG. 9-C shows an example of a shaping cutting path 230 of a shaping through cut which provides different round trip paths which intersect, resulting in the shaping and separation of a plurality of blades 30. In accordance with a possible operating mode, the cutting profile 230 shown in FIG. 9-C can be understood as a single return path to the at least one outward path shown in FIG. 9-B, and in such a case the single return path machines substantially straight edges of the body of the blades 30 and the shaping through cut performed along said single return path of the shaping cutting path 230 performs the function of separating the blades 30. In accordance with a possible operating mode, the cutting profile 230 shown in FIG. 9-C can be understood as a shaping cutting profile independent from the one shown in FIG. 9-B and the round trip path can be chosen if necessary.

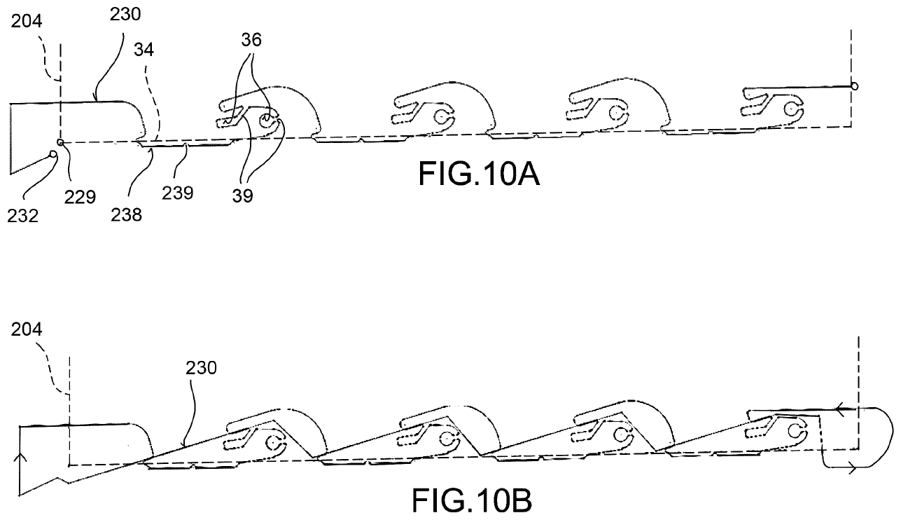

FIGS. 10-A and 10-B show an example similar to that shown in FIGS. 9-B and 9-C described above.

The sharpening cutting path can be performed multiple times i.e., with multiple repeated passes, e.g., round trip passes, e.g., in a number between 3 and 11 passes, and preferably between 3 and 7 passes. In accordance with an operating mode, said sharpening cutting path of the sharpening step is performed more often than the shaping cutting path of the shaping. This results in a better finish of the sharp edge 34. In accordance with a preferred operating mode, the sharpening cut is performed before the shaping cut so that during the process of making the blade the piece is not subjected to vibrations during the first or the multiple finishing passes.

The shaping cut is preferably also detaching, i.e., it results in the separation of the blade 30, and is preferably performed after making the blade and preferably in a single pass.

In accordance with a possible operating mode, the sharpening step is performed by a single cutting sharpening trajectory 240 of the cutting wire 202 and said shaping step is performed by a single cutting shaping trajectory 230 of the cutting wire 202. Preferably, the sharpening cutting path or trajectory 240 has a starting point 235 and an ending point 236, which can be coincident if an even number of round trip passes are performed. Preferably, the shaping cutting path or trajectory 230 has a starting point 232 and an ending point 233, which can be coincident in the case in which an even number of round trip passes are performed.

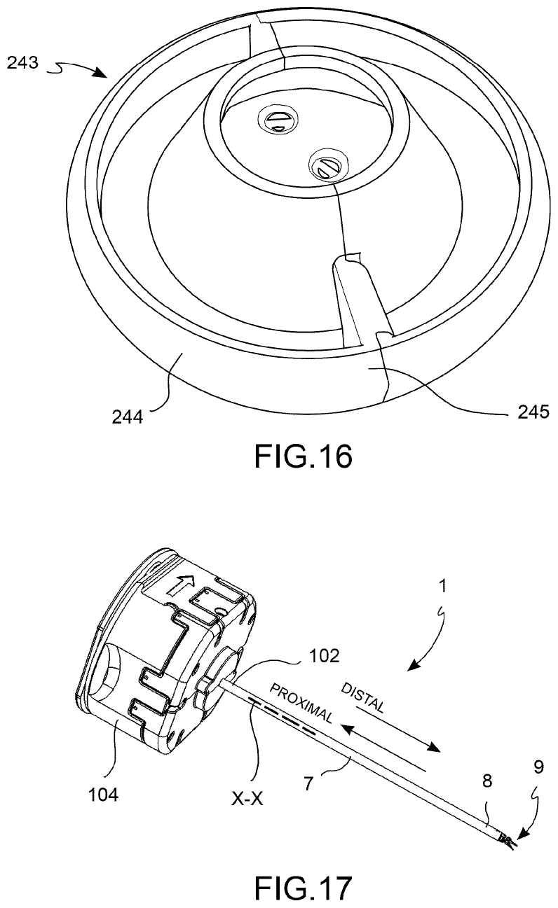

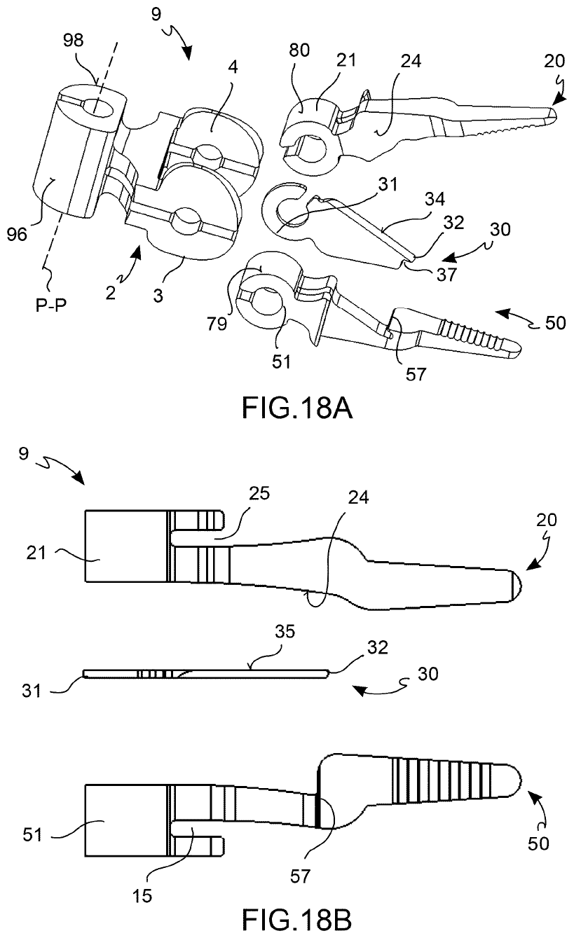

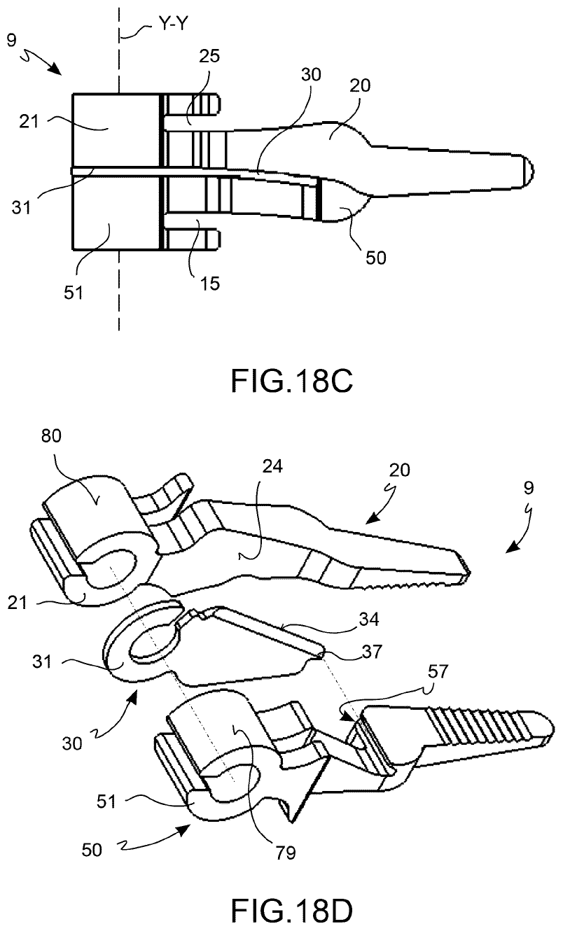

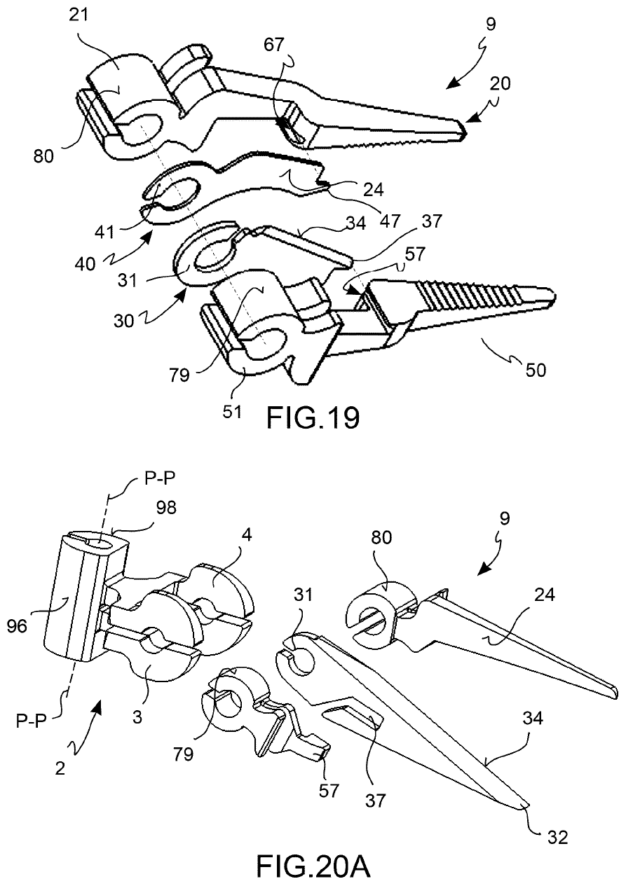

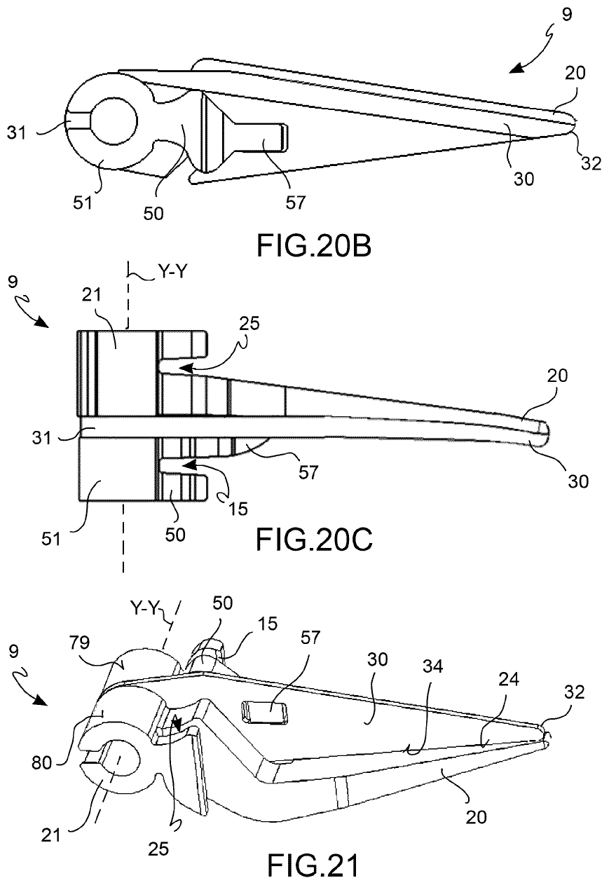

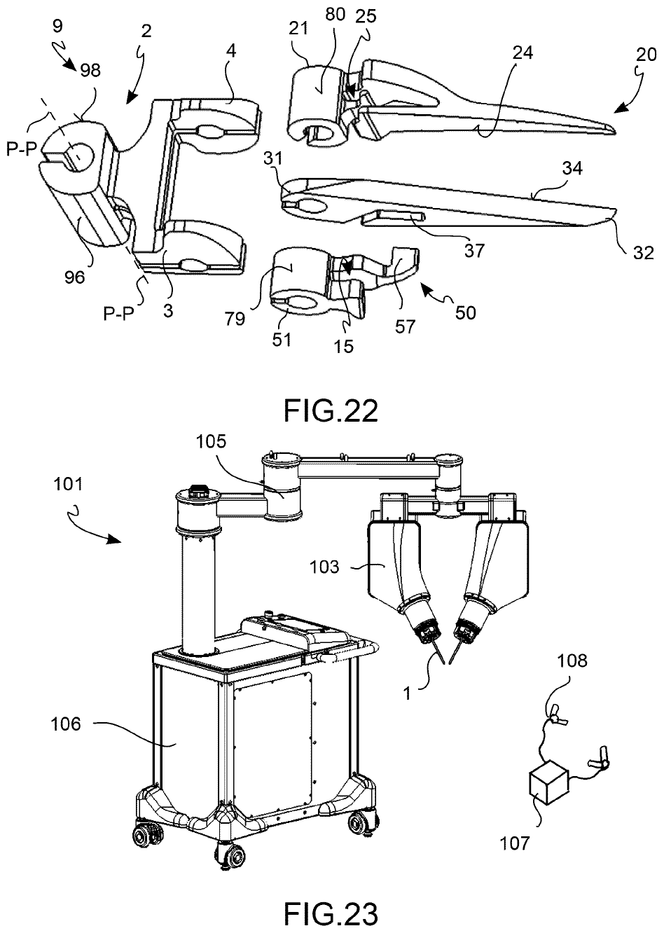

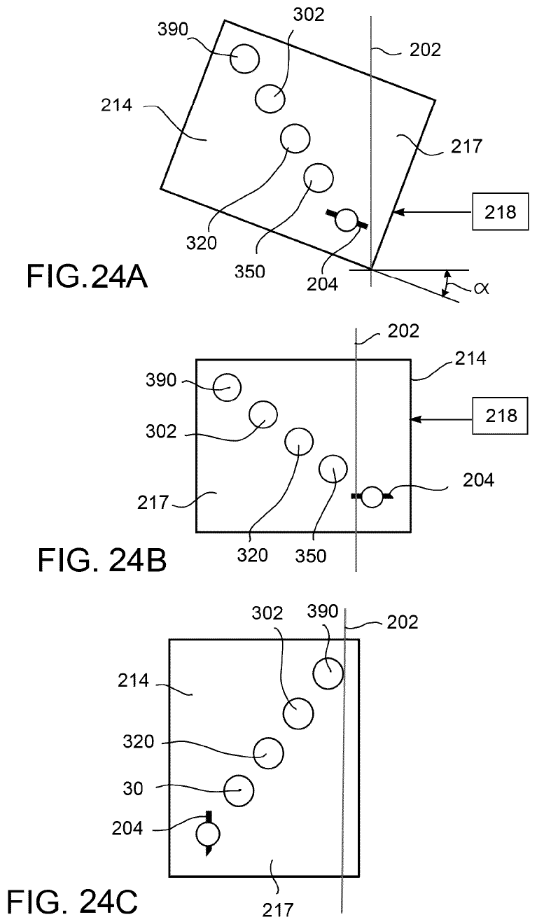

A basket 243 can be provided for collecting the blades 30 which are separated, as shown for example in FIG. 16. For example, the basket 243 is made of two separable half-bodies 244, 245 which can be assembled, for example interlocked, around the lower head 206 of the electro-erosion machine 200, forming when assembled at least one collection chamber having a substantially annular shape to collect the separate blades 30 which, due to the effect of gravity, fall into the dielectric liquid tank 208. In such a case, the method can comprise, after the step of separating the blades 30, the step of collecting by gravity the sharpened, shaped and separated blades 30 by wire electro-erosion.

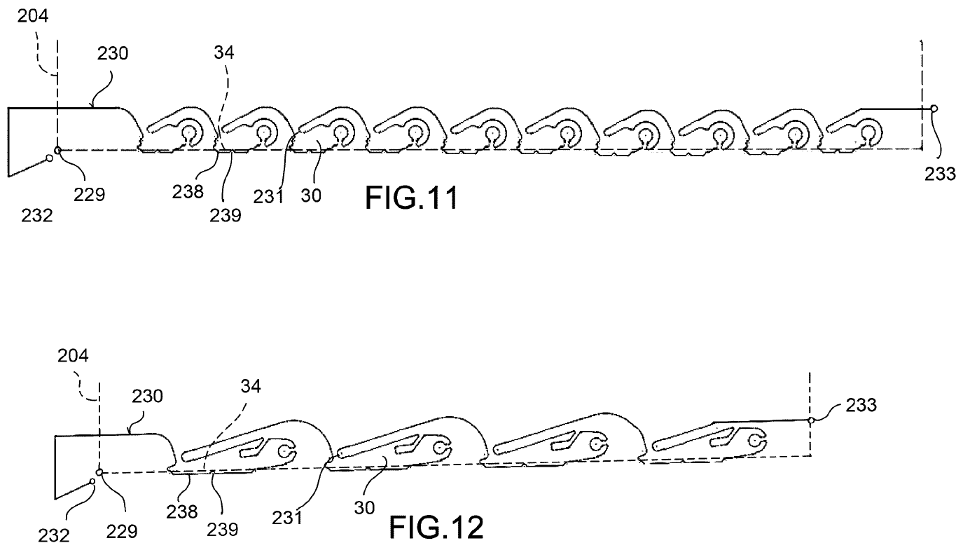

FIG. 11 and FIG. 12 each show an example of a shaping cutting path 230 of a shaping through cut which describes the shape of a plurality of blades 30 on a same workpiece, each provided with a connection bridge 231, making undercuts, hole edges 36, passage channels 39, said external section 238 with respect to the sharp edge 34. The shaping cutting path 230 shown here can be performed several times, i.e., with multiple repeated passes, for example round trip passes. In such a case, the method can include the step of separating the blades 30 comprising breaking the breakable connection bridges 231 to be performed elsewhere and for example the step of separating the blades by breaking the connection bridges 231 can be carried out during the assembly of the finished product, such as a surgical cutting instrument.

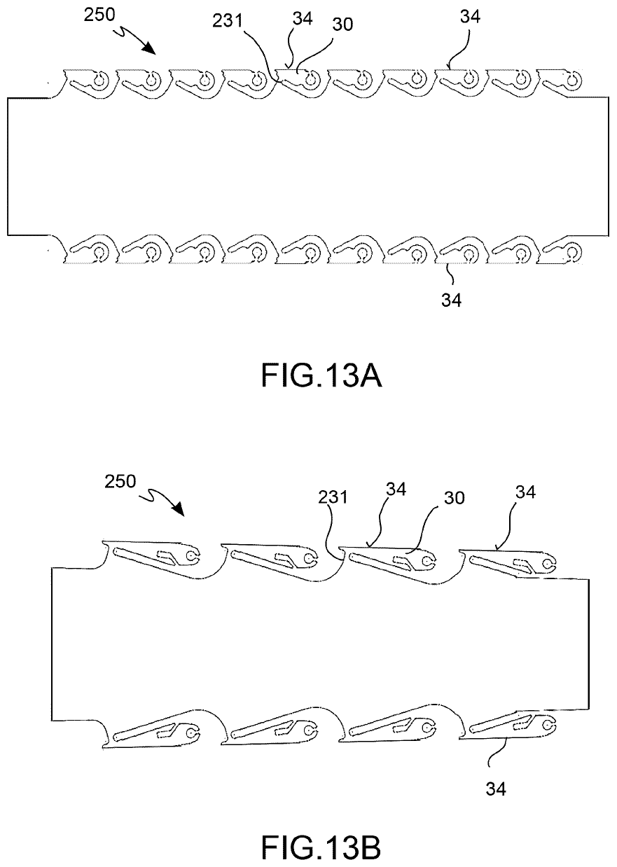

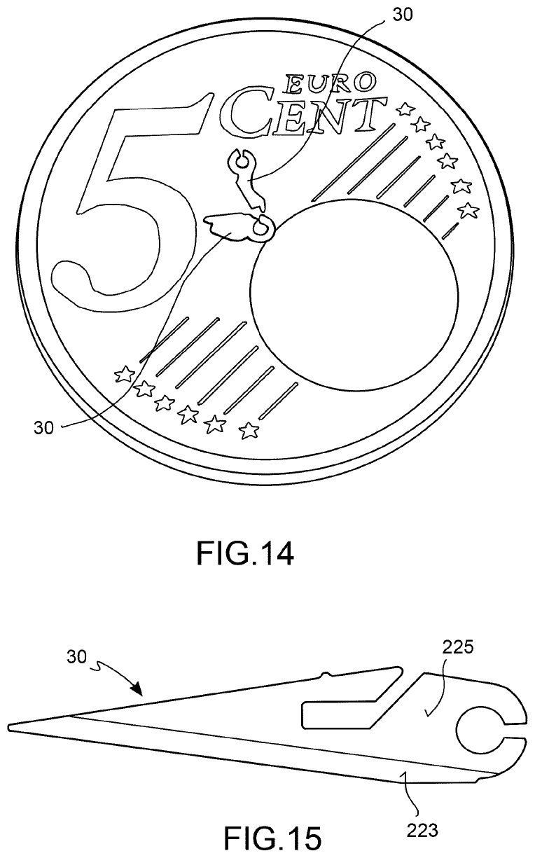

FIGS. 13-A and 13-B show some examples of a semi-finished product 250 made with a method according to any one of the operating modes described herein comprising a plurality of blades 30 each provided with a connection bridge 231, for example made of breakable material. In accordance with an operating mode, the method further comprises the step of making said semi-finished product 250 and the step of separating the blades 30 by breaking the respective connection bridges 231.

The step of breaking the connection bridges 231 can be performed by wire electro-erosion, making a shaping cut.

In accordance with a possible operating mode, first the shaping step is performed, then the rotation step, then the sharpening step. Thereby, the shaping is done first, and the shaping after.

This possible operating mode is preferably performed if the connection bridge, the shape of the piece or the thickness of the piece itself are sufficient not to induce vibrations during the one or more sharpening passes on the already shaped piece.

In accordance with a possible operating mode, the shaping step is performed first, then the rotating step, then the sharpening step, then a further rotating step and then a further shaping step, i.e., the shaping step can be partially performed before the sharpening step and completed after the sharpening step. In accordance with this operating mode, the shaping step can leave the shapes of the one or more blades traced by cutting on the workpiece but interconnected by bridges of material 231, for example breakable bridges of material of locally reduced thickness.

In accordance with an embodiment, the method determines making a semi-finished product 250 comprising a plate-like body in which a plurality of blades 30, each having a sharp edge 34 in which the blade bodies are interconnected together by one or more material bridges 231 of the workpiece body which has not been intentionally removed, for example breakable material bridges.

In accordance with a possible operating mode in which the shaping step is performed first, followed by the rotating step, then the sharpening step, and in which the shaping step makes shapes on the workpiece 204 of the one or more cuttingly shaped blades (but without a sharp edge 34) and interconnected by material bridges 231, the sharpening step can be performed on the edges to be sharpened 234 of the individual blade shapes, although the cutting path can still follow a continuous path which in some sections does not cross material of the workpiece which has already been removed, for example, from the shaping through cut.

In accordance with a possible operating mode, the sharpening and shaping steps can alternate and a rotation step is always included therebetween.

Multiple sharpening cuts on different cutting planes and/or multiple shaping cuts on different cutting planes can be included. For example, a step of rotating the jig between two adjacent sharpening steps can be included, and/or a step of rotating the jig two adjacent shaping steps can be included. For example, between two shaping cuts of the same workpiece, a rotation angle of the jig 214 of substantially 90° can be included, even if between said two shaping cuts a sharpening cut at another, further orientation is included.

For example, between two sharpening cuts of the same edge to be sharpened of the same workpiece, a rotation angle of the jig 214 greater than or equal to 90° can be included, albeit in order to make an acute angle β in the body of the workpiece 204. In accordance with a possible operating mode, two sharpening through cuts are made on two cutting planes rotated therebetween by 90°-150° and preferably 120°-150°.

In accordance with a possible operating mode, the method comprises the step of separating said one or more blades 30. The separating step can be included in the shaping step, where the cutting path of the shaping through cut makes one or more separate blades. Where a semi-finished product 250 is produced in which a plurality of blades 30 each having a sharp edge 34 in which the blade bodies are interconnected by one or more material bridges 231 is cuttingly shaped, the separating step can comprise breaking said material bridges 231 and could also be performed at the assembly site.

In accordance with a possible operating mode, the workpiece 204 is an elastic body having an elastically deformable body for exerting an elastic reaction. In accordance with an embodiment, the workpiece 204 is an elastic plate-like body, for example it is an elastic strip adapted to bend elastically. The provision of an elastically bendable workpiece allows making a miniaturized elastic blade having an elastically bendable body.

Preferably, the workpiece 204 is made of metallic material. The workpiece 204 can be made of steel for blades. One or more surface treatments 228 on the workpiece can be included, such as coatings and/or heat treatments, for example to make the cutting edge 34 harder and more resistant to wear when in operating conditions. In accordance with an embodiment, the cutting edge 34 comprises a surface treatment 228 at least on the surface intended to work by mechanical interference contact against a counter-blade when in operating conditions.

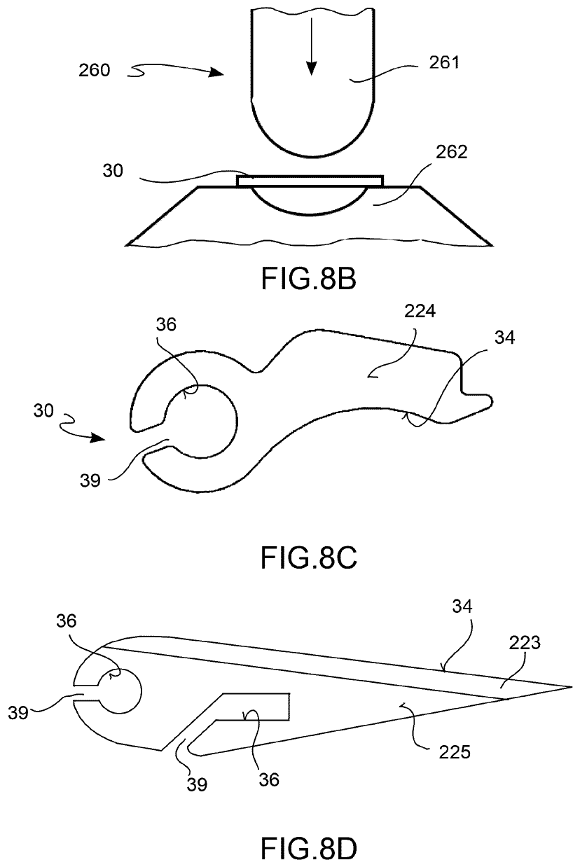

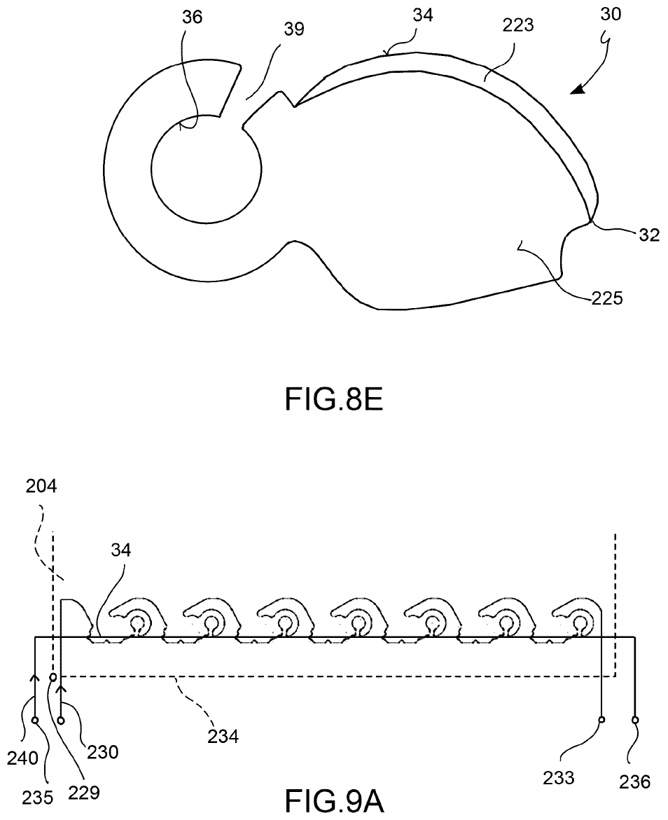

The workpiece 204 can be subjected to bending such as by press-bending, for example as shown in FIG. 8-B. In such a case, the method comprises the step of bending the blade 30. This step can comprise the step of including a press 260, for example having a hammer 261 and an anvil 262. Bending by press-bending can be performed to give the blade elastic properties.

In accordance with a possible operating mode, the method comprises the step of treating the surface of the workpiece, obtaining a surface treatment 228 on the workpiece. The step of treating the surface can also be performed more than once.

In accordance with a possible operating mode, the step of treating the surface is performed before the sharpening step. Where the surface treatment 228 is carried out before said sharpening step, then the wall 223 exposed by the flush cut of the cutting edge 34 will lack surface treatment 228. In this case, for example, a “no-back-bevel” or “chisel edge” type sharpening can be obtained in which the surface 35 of the cutting edge 34 intended to work by mechanical interference contact against a counter-blade when in operating conditions comprises a surface treatment 228 while the opposite cutting wall 223 does not comprise any surface treatment 228.

In accordance with a possible operating mode, the step of treating the surface is performed after the sharpening step. Where the surface treatment 228 is carried out after said sharpening step, then the wall 223 exposed by the flush cut of the cutting edge 34 can comprise a surface treatment 228.

In accordance with a possible operating mode, the step of treating the surface comprises the step of making a diamond-like-carbon (DLC) type coating or the like.

In accordance with a possible operating mode, the step of treating the surface comprises the step of carrying out a heat treatment, for example of the “Kolsterizing®” type or the like.

In accordance with an operating mode, the step of coating the surface is performed when the workpiece is in the form of a semi-finished piece 250 having a body comprising in a single piece a plurality of sharp bodies, for example a plurality of blades, shaped and interconnected by connection bridges 231. Thereby, the miniaturization of the sharp bodies is facilitated because it allows positioning a plurality of sharp bodies together for surface treatment, by positioning the body of the semi-finished piece 250, for example a ribbon or strip.

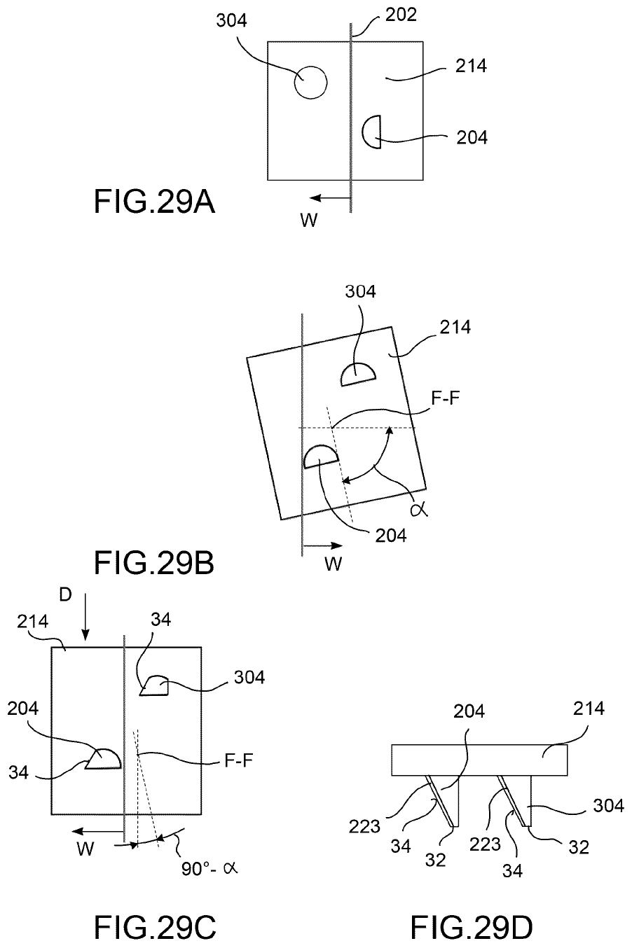

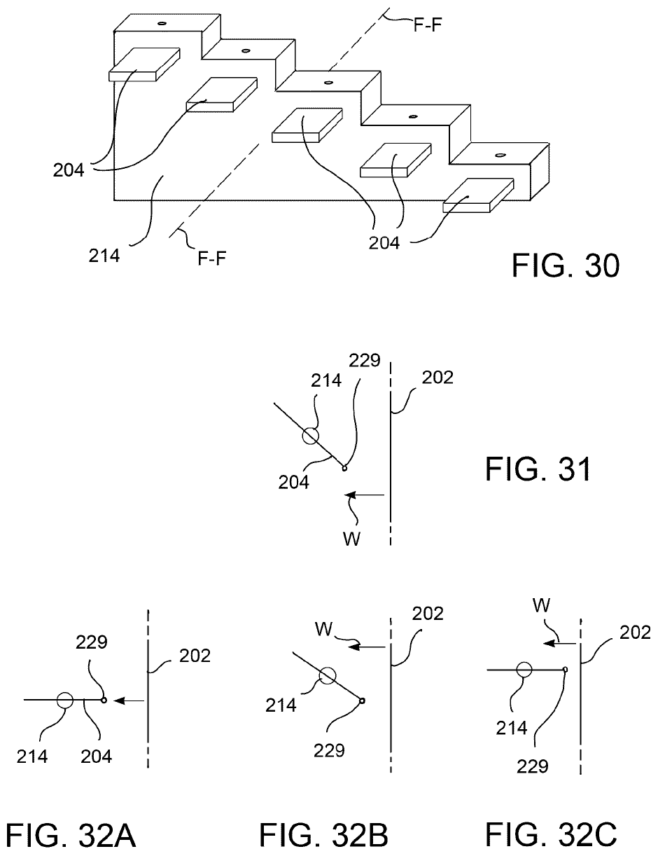

In accordance with a possible operating mode, the method further comprises after the shaping step, the further reshaping step to make a second shaping, on a second cutting plane, said workpiece 204, performing with the cutting wire 202 a second shaping through cut on the at least one workpiece 204, in which between the shaping step and the reshaping step, the step of rotating said fixture 214 by a shaping angle preferably substantially equal to 90° is included. In accordance with this operating mode, preferably the shaping step is performed before the sharpening step. As shown, for example, in the sequence of FIGS. 29A-C, it is possible to carry out first the shaping step, followed by the sharpening step, then the reshaping step, in which between the shaping step and the reshaping step the workpiece 204 has been rotated by rotation of the fixture or a portion thereof at an angle substantially equal to 90°.

Between the shaping step and the sharpening step, the workpiece 204 can be rotated by a sharpening angle α.

Thereby it is possible to perform two shaping cuts and one sharpening cut on the same workpiece 204.

In accordance with a possible operating mode, the mounting step comprises mounting on said fixture 214 a plurality of workpieces 204, 304, and in which the sharpening and shaping steps comprise individually sharpening and shaping each workpiece. In other words, in accordance with this operating mode, each workpiece 204, 304 is machined individually, avoiding performing simultaneous cuts on a multiplicity of workpieces. Where different cuts are made on different pieces, said cuts can be made in succession on the different pieces.

In accordance with a possible operating mode, the mounting step comprises mounting on said fixture 214 also at least a second workpiece 304 so as to obtain at least two workpieces 204, 304 mounted on the same fixture 214, and in which the method further comprises sharpening at least one edge to be sharpened of said second workpiece 304, and in which between the sharpening step at least one edge to be sharpened of the at least one workpiece 204 and the step of at least one edge to be sharpened of said second workpiece 304 a further step of rotating at least one portion of said fixture 214 is included. Thereby it is possible to obtain different sharpnesses on different workpieces 204, 304.

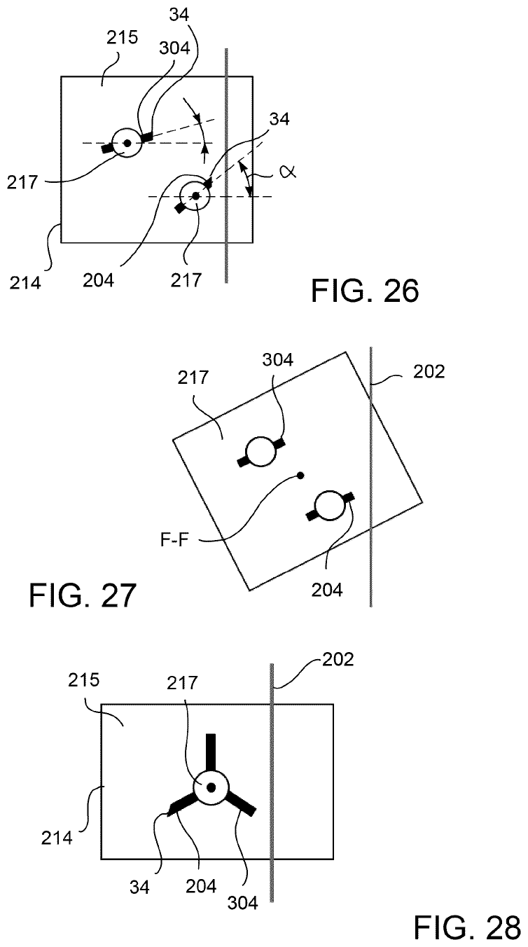

As shown for example in FIG. 26, two sharpening cuts can be made on different workpieces by rotating the housing portion 217 which mounts each workpiece 204, 304 by a different sharpening angle, i.e., a rotation of a first workpiece 204 by a first sharpening angle α and a rotation of a second workpiece 304 by a second sharpening angle α2. Thereby, it is possible to make sharp edges having different acute angle β on different workpieces 204, 304.

As shown for example in FIG. 27, it is possible to make two sharpening cuts on different workpieces 204, 304 integral in rotation with each other by providing between two sharpening steps, i.e., between the step of sharpening at least one edge to be sharpened of the at least one workpiece 204 and the step of at least one edge to be sharpened of said second workpiece 304, a further step of rotating at least one portion of said fixture 214 by a certain angle, for example equal to α2-α. The angles α and α2 can be different from each other by any amount. The angle α2 can be chosen according to the same considerations set forth with reference to the angle α and thus with reference to the direction of the cutting wire 202 for performing a shaping cut.

In accordance with a possible operating mode, the fixture 214 receives a plurality of workpieces 204 having a plate-like body arranged so as to be individually and singularly machinable by the cutting wire 202, in one or more rotation configurations of the fixture 214.

As shown for example in FIG. 28, three (or more) workpieces 204 having a plate-like body can be star-shaped on the fixture 214. i.e., they can be arranged to extend with a respective cantilevered flap from the housing portion 217 of the fixture 214 in radial directions with respect to the housing portion 217. For example, the workpieces in star configuration can be individually sharpened and between the sharpening of one workpiece and another, a step of rotating the housing portion 217 of the fixture 214 can be included.

In accordance with an embodiment, the fixture 214 or jig 214 includes fixing multiple planar elements (strips), which can be machined individually by electro-erosion in one or more rotation configurations.

In accordance with a possible operating mode, the method comprises at least two shaping steps, i.e., a shaping step and a reshaping step, and between said two shaping steps the further step of rotating the jig 214 by a shaping angle which is preferably substantially equal to 90° is included. In other words, preferably, the two shaping steps are performed on two cutting planes orthogonal to each other. It is also possible for the method to first provide a first shaping step, then rotate the jig 214 by said sharpening rotation angle α (e.g.: α=40° and perform a sharpening step, then rotate the jig 214 again by an angle equal to 90°-α (in this example, therefore 50°) and perform a second shaping step, in which from the first shaping step to the second shaping step the jig 214 has rotated by 90°. This operating mode can be advantageous for producing with a single placement of the workpieces in the electro-erosion machine 200 a link assembly to be assembled together of an articulated end-effector of a surgical cutting instrument (e.g., a surgical scissor or needle-driver/scissors), in which at least one of the links of the link assembly has a sharp edge 34 and for example is a blade link 30.

Accordingly, a method of manufacturing a plurality of links of an articulated end-effector 9 actuatable by actuation tendons for a surgical cutting instrument 1 for robotic surgery by wire electro-erosion comprises the steps reported below. Preferably, this method makes all the links of the articulated end-effector 9 (e.g., an articulated cuff) of the surgical instrument 1. This method can be employed to make links of an articulated end-effector 9 of a robotic non-surgery arm. This method comprises the steps of:
providing a wire electro-erosion machine 200 comprising a cutting wire (202) and a jig 214 which is rotatable with respect to the cutting wire about a rotation axis F-F which is transverse to the longitudinal extension of the cutting wire; and
mounting a plurality of workpieces 204, 302, 320, 350, 390 all integral in rotation with the jig 214 so that the cutting wire 202 intersects at most one of said workpieces 204, at a time. In other words, the workpieces are mounted on the jig in such an arrangement (for example, they are mutually aligned at a certain distance between two adjacent pieces, or they are arranged on a curved line) that they can be machined by the cutting wire 202 singularly, i.e., individually, avoiding cutting more than one workpiece at the same time. Said plurality of workpieces can comprise pieces to be shaped 302, 320, 350, 390 intended to be shaped on two cutting planes and not sharpened, and workpieces 204, 304 intended to be sharpened and also shaped. The pieces to be shaped 302, 320, 350, 390 can be cylinders which are mounted on the jig 214 so that they protrude cantilevered, for example in a direction parallel to the rotation axis F-F.

This method further comprises the steps of:
sharpening at least one edge to be sharpened 234 of at least one workpiece 204 of said plurality of workpieces by performing a sharpening through cut with the cutting wire 202 on the at least one workpiece 204; and
shaping on a first cutting plane at least some, and preferably all, of the workpieces 204, 302, 320, 350, 390 of said plurality of workpieces by performing a shaping through cut with the cutting wire 202 on at least some, and preferably all, of the workpieces, one at a time in succession;
in which between the sharpening step on a first cutting plane and the shaping step the further step is performed of:
rotating the jig 214 about the rotation axis F-F thereof by a sharpening rotation angle α other than 90° in absolute value (with regard to the sharpening angle α one or more of the considerations described above may apply);
reshaping on a second cutting plane at least some, but also all, of the workpieces 302, 320, 350, 390 of said plurality of workpieces by performing a shaping through cut with the cutting wire 202 on said at least some workpieces of said plurality, one at a time in succession.

Between shaping step on a first cutting plane and the shaping step on a second cutting plane, the step of rotating the jig 214 about the rotation axis F-F thereof by a rotation angle substantially equal to 90° is included. As explained above, depending on the order which can be arbitrarily chosen of the steps of sharpening, shaping on a first cutting plane and shaping on a second cutting plane, this rotating step by a rotation angle substantially equal to 90° can be operatively performed in two moments, in which one of the two execution moments corresponds to the step of rotating the jig 214 about the rotation axis F-F by a sharpening rotation angle α.

The arrangement of the workpieces of said plurality of pieces to be machined on the jig preferably must meet the condition that the cutting wire 202 intersects at most one of the workpieces at a time in each step (sharpening, first shaping, second shaping). For example, where only one of the workpieces is to be subjected to the sharpening step, such a workpiece 204 can be arranged at the edge of a row according to which the workpieces of the plurality of workpieces are arranged.

The housing portion 217 of the jig 214, i.e., the part of the jig which is rotatable with respect to the fixing portion 215, in this embodiment preferably comprises a plurality of housing seats 241 integral in rotation with one another. Preferably, the housing seats 241 are mutually aligned.

In accordance with a possible operating mode, the shaped pieces as well as the sharpened pieces are assembled together. Therefore, the method can comprise the step of assembling the pieces obtained together.

In accordance with a possible operating mode, the shaping step and/or the reshaping step comprises shaping two workpieces differently. In accordance with a possible operating mode, the shaping step comprises shaping two workpieces so that one portion of a shaped piece is complementary to one portion of another shaped piece.

In accordance with a possible operating mode, the rotating step comprises providing a rotating support table and rotating said rotating support table. The rotating support table is preferably integral with at least one and preferably all the workpieces.

In accordance with a possible operating mode, the method is performed by providing at least some workpieces of said plurality in the form of material cylinders, for example said pieces to be shaped 302, 320, 350, 390 are material cylinders which are mounted on the jig 214 so that they protrude cantilevered and in which the shaping and reshaping steps create 90° edges on said cylinders. In other words, the shaping and reshaping steps remove material from the curved side face of the cylinders, creating orthogonal faces.

In accordance with a possible operating mode, the method makes three links of the articulated end-effector to be assembled together, in which at least one link is a link comprising a sharp edge 34, and the housing portion 217 of the jig 214 comprises three housing seats 241 integral in rotation with one another. For example, said three links are: said blade link 30 having said cutting edge 34, said blade holder link 50 and said second tip link 20 comprising said counter-blade surface 24.

It is also possible that two links are obtained from a single workpiece and in such a case the method can make a plurality of links of the articulated end-effector 9 to be assembled together, in which at least one link is a link comprising a sharp edge 34, and the housing portion 217 of the jig 214 comprises at least two housing seats 241 integral in rotation with each other. For example, the blade holder link 50 and the second tip link 20 can be manufactured from the same workpiece.

In accordance with a possible operating mode, the method makes five links of the articulated end-effector to be assembled together, in which at least one link is a link comprising a sharp edge 34, and the housing portion 217 of the jig 214 comprises five housing seats 241 integral in rotation with one another. Where two links are obtained from a single workpiece and in such a case the method makes five links of the articulated end-effector 9 to be assembled together, in which at least one link is a link comprising a sharp edge 34, and the housing portion 217 of the jig 214 comprises at least two housing seats 241 integral in rotation with each other.

Preferably, the at least one workpiece 204 is machined by sharpening and one shaping and the other workpieces 302, 320, 350, 390 are not machined by sharpening, so that each workpiece is machined with two through cuts on two different cutting planes, without disassembling the pieces between one cut and another, in which the through cuts are not the same for all the pieces because at least the sharpening cut on the at least one piece 204 has a different inclination than both shaping cuts performed globally.

In accordance with a general embodiment, a semi-finished product 250 is provided comprising a sheet-like body in a single piece having a plurality of sharp shaped bodies connected together by one or more breakable connection bridges 231. The semi-finished product 250 can comprise any one of the features described with reference to any one of the embodiments described above.

The semi-finished product 250 can comprise a surface treatment 228 or can be intended to receive a surface treatment 228.

In accordance with a general embodiment, a fixture 214 or jig 214 is provided for an electro-erosion machine 200.

Said fixture 214 or jig 214 comprises a fixing portion 215 for mounting the fixture 214 to the electro-erosion machine 200 and a housing portion 217 for receiving at least one workpiece 204, in which the housing portion 217 is rotatable with respect to the fixing portion 215 about a rotation axis F-F.

Preferably, the fixture 214 further comprises a motor 218 for rotating the housing portion 217 with respect to the fixing portion 215.

The fixture 214 or jig 214 can comprise any one of the features described with reference to any one of the embodiments described above.

In accordance with an embodiment, the housing portion 217 of the fixture 214 comprises a plurality of seats for receiving a plurality of workpieces, in which the seats for said plurality of workpieces are arranged so that two orthogonal lines intersect one workpiece at a time. In other words, the seats are arranged so that when the workpieces are mounted on the jig 214, the cutting wire 202 of the electro-erosion machine 200 cuts only one of said workpieces on two orthogonal cutting planes. Preferably, the seats for said plurality of workpieces are arranged so that three lines, two lines of which orthogonal to each other and a third one inclined by a sharpening angle α, intersect only one workpiece at a time. For example, the seats are arranged on the fixture 214 so as to be mutually aligned at a certain relative distance.

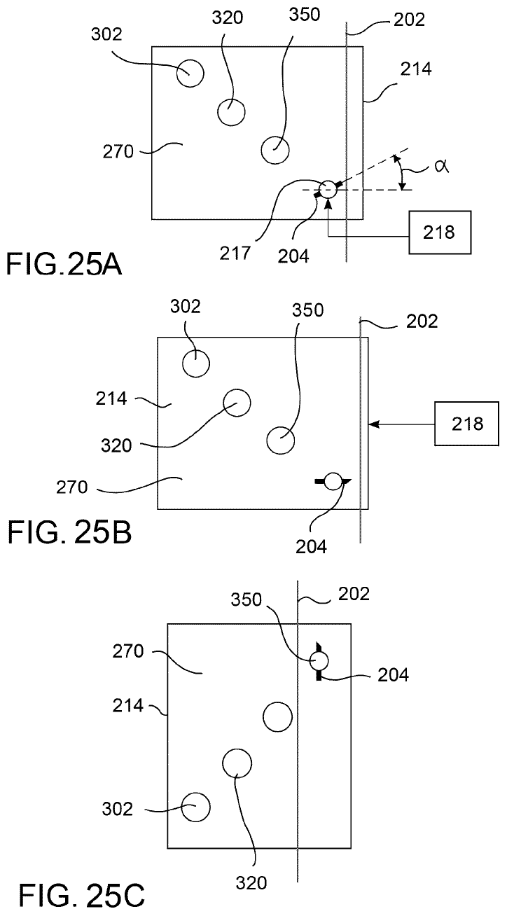

In accordance with an embodiment as diagrammatically shown in FIGS. 25A-C, the jig 214 comprises two housing portions 217, 270 which are individually or jointly rotatable with respect to the fixing portion 215 to the machine 200, in which a first housing portion 217 receives said workpiece 204 to make a sharpening cut and a shaping cut thereon, and a second housing portion 270 receives both said first housing portion 217 and one or more further workpieces 302, 320, 350 to make two orthogonal shaping cuts thereon. Preferably, the first housing portion 217 is mounted to the second housing portion 270 so that it can rotate with respect to said second housing portion about a rotation axis F-F. A single motor 218 for obtaining the rotations of the first housing portion 217 and of the second housing portion 270 can be included.

In accordance with a general embodiment, a surgical cutting instrument 1 is provided. For example, said surgical cutting instrument 1 is a surgical scissor type instrument. For example, said surgical cutting instrument 1 is an instrument of the needle-driver/suture-cutter type.

The surgical instrument 1 preferably comprises a shaft 7 having a distal end 8 and an articulated end-effector 9 (in other words an articulated end device 9 connected to the distal end 8 of the shaft 7.

Said surgical instrument 1 is particularly suitable, but not uniquely intended, for robotic surgery and can be connectable to a robotic manipulator 103 comprising motorized actuators of a robotic surgery system 101, as for example shown in FIG. 23. For example, said surgical instrument 1 can be associated with a mechanical and manual control and actuation device.

The robotic surgery system 101 comprising said surgical instrument 1 is particularly suitable, but not uniquely intended, for robotic microsurgery operations. The robotic surgery system 101 can be intended for robotic laparoscopy operations.

Not necessarily said shaft 7 is a rigid shaft and for example can be a bendable shaft and/or an articulated shaft, although in accordance with a preferred embodiment said shaft 7 is a rigid shaft. A proximal interface portion 104 or backend portion 104 of the surgical instrument 1 can be provided at the proximal end 102 of the shaft 7, to form the interface with a robotic manipulator 103 of the robotic surgery system 101, as shown for example in FIG. 17. A sterile barrier can be interposed between the robotic manipulator and the proximal interface portion 104 of the surgical instrument. For example, said proximal interface portion 104 can comprise a set of interface transmission elements for receiving the driving actions imparted by the robotic manipulator 103 and transmitting them to the articulated end-effector 9. In accordance with an embodiment, the surgical instrument 1 is detachably associated with the robotic manipulator 103 of the robotic surgery system 101.

The articulated end-effector 9 at the distal end 8 of the shaft 7 can comprise a plurality of links articulated to one another in one or more rotational joints movable by a number of pairs of antagonistic actuation tendons extending from the proximal interface portion 104 to the articulated end-effector 9 inside the shaft 7 ending in termination seats 15, 25 provided on at least some of the links of the articulated end-effector 9. The pair of actuation tendons of one or more pairs of antagonistic tendons can be obtained with a single tendon forming a round trip path from the proximal interface portion 104 of the instrument to a link of the articulated end-effector of the instrument.

Preferably, the term “link” refers to a body made in a single piece, i.e., a monobloc body.

Preferably, each of the links of the articulated end-effector 9 is made by a method according to any one of the previously described operating modes.

Not necessarily all the links forming the articulated end-effector 9 are articulated, i.e., movable, with respect to one another and/or with respect to the distal end 8 of the shaft 7. For example, said end-effector 9 can be an articulated cuff of the “roll-pitch-yaw” type according to a terminology widely adopted in the field. For example, said end-effector 9 can be an articulated end-effector 9 of the “snake” type, i.e., comprising a multitude of coplanar and/or non-planar rotational joints.

In accordance with an embodiment, said articulated end-effector 9 comprises a connection link connected to the distal end 8 of the shaft 7 having a body comprising in a single piece one or more convex ruled surfaces of connection links with parallel generatrices. The connection link further comprises in a single piece a first distal connecting portion. Preferably, said first distal connecting portion of the first connection link comprises two prongs and is adapted to form a proximal rotational joint having a proximal rotation axis P-P. In accordance with a preferred embodiment, the convex ruled surfaces generatrix lines of the connection link are all parallel to the proximal rotation axis P-P.

In accordance with a preferred embodiment, said articulated end-effector 9 comprises a support link 2 articulated to the connection link and having a body comprising in a single piece one or more convex ruled surfaces of support links 96, 98 with parallel generatrices. The support link 2 further comprises in a single piece a proximal connecting portion articulated to the first distal connecting portion of the first connection link, defining a proximal rotational joint for the connection link and the support link 2 so that they can rotate relatively about a common proximal rotation axis P-P.

The support link 2 further comprises in a single piece a distal connecting portion. The distal connecting portion of the support link 2 preferably comprises a support structure, for example comprising two prongs 3, 4, for defining a distal rotation axis Y-Y, i.e., for forming a distal rotational joint or yaw rotational joint having a common distal rotation axis Y-Y, or yaw axis Y-Y, which can be orthogonal to the pitch proximal rotation axis P-P.

The support structure of the support link 2 is preferably a rigid support structure, i.e., it is for example a rigid support fork, the relative position of the prongs 3, 4 is rigidly determined as is the relative position of a prong 3, 4 and a ruled surface 96, 98. In accordance with an embodiment, said distal rotation axis Y-Y is a yaw rotation axis Y-Y and said proximal rotation axis P-P is a pitch rotation axis P-P, in which the yaw rotation axis Y-Y and the pitch rotation axis P-P are orthogonal to each other.

In accordance with an embodiment, the articulated end-effector 9 further comprises a blade holder link 50, articulated to the support link 2 having a body comprising in a single piece an attachment root of a blade holder link 51 having a pulley portion 79 formed by one or more convex ruled surfaces 79 of blade holder root with parallel generatrices. The blade holder link 50 comprises in a single piece a proximal attachment root 51 which is articulated in said distal rotational joint.

The articulated end-effector 9 preferably comprises a blade link 30, integral in rotation with said blade holder link 50, having a body comprising in a single piece a cutting edge 34. The cutting edge 34 is adapted to perform a cutting action.

The blade link 30 is made by a method according to any one of the previously described operating modes.

In accordance with an embodiment, the blade link 30 comprises in a single piece a proximal attachment root 31 which is articulated in said distal rotational joint. The blade link 30 preferably comprises in a single piece an attachment root 31 arranged side by side with the root 51 of the blade holder link 50 and preferably the root 31 of the blade link 30 is side by side in direct and intimate contact with the root 51 of the blade holder link 50.

In accordance with an embodiment, the body of the blade holder link 50 further comprises in a single piece a drag portion 57 and the body of the blade holder link 30 further comprises in a single piece a drag counter-portion 37 engaged with said drag portion of the blade holder link 50. The drag engagement can be obtained by an engagement between the blade link 30 and the blade holder link 50. The drag engagement between the blade link 30 and the blade holder link 50 can be arranged distally with respect to the common rotation axis Y-Y. i.e., distally with respect to the attachment roots 31 and 51. In such a case, the drag engagement portion 37 (or drag portion 37) of the blade link 30 is preferably positioned far from the blade link root 31 so as to ensure a precise drag, even though the drag portion 37 of the blade link 30 can be positioned at the blade link root 31 to achieve a more advantageous mechanical transfer.

In accordance with an embodiment, the articulated end-effector 9 further comprises a reaction link 20 articulated to the support link 2 and to the blade holder link 50, the reaction link 20 having body comprising in a single piece a further attachment root 21 of reaction link with a pulley portion 80 formed by one or more convex ruled surfaces with parallel generatrices.

In accordance with an embodiment, the support link 2, the group formed by the blade holder link 50 and the blade link 30, and the second tip are articulated to one another in said common rotation axis Y-Y defining an axial direction coincident with or parallel to the common rotation axis Y-Y. In other words, the distal connecting portion 17 of the support link 2 is articulated with respect to the group formed by the root 51 of the blade holder link 50 and the root 31 of the blade link 30, and the root 21 of the reaction link 20 in said distal common rotation axis Y-Y. Preferably, for clarity of presentation, an axial direction coincident or parallel with the direction of the common rotation axis Y-Y is defined.

Preferably, for clarity of presentation, with reference to the blade link 30 and/or the blade holder link 50, an internal axial direction facing along the axial direction towards said fifth is also defined, further reaction link 20 and similarly said internal axial direction will be with reference to the reaction link 20 facing opposite i.e., towards the blade link 30 and/or blade holder link 50.

The proximal and distal directions (or senses) are understood as referring in accordance with the common meaning of the terms, as shown by the arrows in FIG. 17.

Preferably, for clarity of presentation, the term “radial” will refer to a direction which is substantially orthogonal to the common rotation axis Y-Y and incident thereto.

Preferably, for clarity of presentation, it also means a longitudinal direction which globally can be substantially coincident with the longitudinal extension direction of the surgical instrument 1, as well as locally with the longitudinal extension direction of the elongated body of the blade link 30 and/or the blade holder link 50 and or the reaction link 20.

In accordance with an embodiment, the root 21 of the reaction link 20 and the group formed by: the root 51 of the blade holder link 50 and the root 31 of the blade link 30 are articulated with respect to the distal portion of the support link 2 about said common rotation axis Y-Y defining a degree of freedom of orientation of yaw Y. Therefore, the common rotation axis Y-Y (or a straight extension thereof) crosses said two prongs 3, 4, and said roots 21, 31, 51 and can be defined by an articulation pin.

In accordance with an embodiment, moreover, the root 21 of the further fifth reaction link 20 is articulated with respect to the group formed by: the root 51 of the blade holder link 50 and the root 31 of the blade link 30 about said common rotation axis Y-Y, defining a relative degree of freedom of opening/closing G (or degree of freedom of cutting G, or degree of freedom of grip G according to a widely adopted terminology, although the activation of this degree of freedom does not necessarily result in a gripping action) to exert the cutting action.

In accordance with an embodiment, a counter-blade portion 24 is provided which is integral in rotation with said attachment root 21 of the reaction link 20. Therefore, the reaction link 20 is integral in rotation with the counter-blade portion 24. Not necessarily, the reaction link is in a single piece with the counter-blade portion 24, although in accordance with a preferred embodiment the reaction link 20 comprises the attachment root 21 and the counter-blade portion 24 in a single piece.

In accordance with an embodiment, the surgical cutting instrument 1 further comprises a first pair of antagonistic tendons extending along the shaft 7 and connected to the blade holder link 50 to move the blade link 30 about said common distal rotation axis Y-Y. The attachment root 51 of the blade holder link 50 comprises in a single piece at least a first termination seat 15 which receives said first pair of antagonistic tendons.

In accordance with an embodiment, the surgical cutting instrument 1 further comprises a second pair of antagonistic tendons extending along the shaft 7 and connected to said further reaction link 20 for moving the counter-blade portion 24 about said common yaw rotation axis Y-Y. The attachment root 21 of the reaction link 20 comprises in a single piece at least a second termination seat 25 which receives said second pair of antagonistic tendons.

Each tendon has a main longitudinal extension and is adapted to work exclusively tensioned.

Each tendon is in contact with the links 2, 20, 30, 50 of the articulated end-effector 9 preferably exclusively on said convex ruled surfaces 79, 80, 96, 98 of at least some of the connection link, the support link 2, the blade holder link 50 (especially the root 51 of the blade holder link 50), the reaction link 20 (especially the root 21 of the reaction link 20). Preferably, the actuation tendons avoid being in contact with the blade link 30, and the blade link 30 is dragged in rotation by the blade holder link 50.

In accordance with an embodiment, said one or more convex ruled surfaces with parallel generatrices of the connection link are parallel to said common proximal rotation axis P-P, and at least one of said one or more convex ruled surfaces 96, 98 with parallel generatrices of the support link 2 is parallel to said common proximal rotation axis P-P. Furthermore, said one or more convex ruled surfaces 79 of the blade holder root 51 with parallel generatrices of the blade holder link 50 and said one or more convex ruled surfaces 80 of the pulley portion of the further root 21 with parallel generatrices of the further reaction link 20 are parallel to the common distal rotation axis Y-Y.

With further advantage, the first pair of antagonistic tendons and the second pair of antagonistic tendons are adapted to longitudinally slide on said one or more convex ruled surfaces of the connection link and on said one or more convex ruled surfaces 96, 98 of the support link 2 and are adapted to wind/unwind without longitudinally sliding on the respective convex ruled surface of the root 79 or 80 of the blade holder link 50 or of the further reaction link 20 to respectively move the blade link 30 and the counter-blade portion 24 in opening/closing.

Therefore, the longitudinal extension of the tendons is locally orthogonal to the lines generating the ruled surfaces on which the tendons are locally in contact.

In accordance with an embodiment, the cutting edge 34 of the blade link 30 is adapted to abut against said counter-blade portion 24 integral in rotation with said reaction link 20, during the movement of the degree of freedom of opening/closing G in a mechanical interference contact condition to exert a cutting action.

In accordance with an embodiment, the cutting edge 34 of the blade link 30 is elastically bendable in a direction parallel to the common distal rotation axis Y-Y. The cutting edge 34 of the blade link 30 is integral in rotation with the first termination seat 15 for the first pair of antagonistic tendons is elastically bendable in the axial direction and said counter-blade portion 24 is adapted to abut against said cutting edge 34, elastically bending the body of the blade link 30 in the axial direction. Thereby, the elasticity in an axial direction for obtaining the cutting action is provided at least partially by the elasticity of the blade portion, while the distal rotational joint 502 to which the root 31 of the blade link 30 is articulated, is axially rigid, i.e., it is not elastically loaded because relative displacements between the distal connecting portion 17 of the support link 2 and the roots 21, 31, 51 of the reaction, blade and blade holder links on the distal rotation axis Y-Y are avoided.

Therefore, said cutting edge 34 of the blade link 30 and said counter-blade portion 24 integral in rotation with the reaction link 20 reach a mechanical interference contact condition to exert a cutting action.

The mechanical interference contact between the cutting edge 34 and the counter-blade portion 24 integral in rotation with the reaction link 20 which results in the cutting action simultaneously bendingly deforms the body of the blade link 30. The bending deformation of the body of the blade link 30 during the cutting action is directed, i.e., it is directed substantially parallel to the common rotation axis Y-Y.

The counter-blade portion 24 preferably comprises a surface facing axially inwards adapted to form mechanical interference contact abutment with the cutting edge 34 of the blade link 30 for axially bending the blade link 30. The reaction link 20 then exerts a reaction in the axial direction to the elastic bending of the blade link 30 during the cutting action. The body of the reaction link 20 can be elastically deformable.

The deformed configuration of the body of the blade link 30 when the blade link 30 and the reaction link 20 are in a substantially closed configuration is maximally bent, and in any case more bent than the configuration of the body of the blade link 30 when the blade link and the reaction link 20 are in a partially closed and partially open configuration. Preferably but not necessarily, when the opening angle is maximally open and the blade is free, the cutting edge 34 is straight and the body of the blade link 30 has a substantially planar configuration.

The at least one point of contact between the cutting edge 34 and the counter-blade portion 24 preferably varies in position and/or size as a function of the opening angle of the degree of freedom of opening/closing G and preferably tends to move in the distal direction as the opening angle is reduced, thereby accentuating the bending by elastic deformation of the body of the blade link 30.

“Point of contact” is preferably meant to indicate the most distal portion of the contact area between cutting edge 34 and counter-blade portion 24, although the contact area can be similar to a point in some configurations of an embodiment.

As described above, the cutting edge 34 can be sharp, i.e., it can be subjected to sharpening in order to have a locally reduced thickness with respect to the thickness of the body of the blade portion 14 and/or a sharp conformation in the cross-section thereof.

During the cutting action, the blade surface 35 of the blade link 30 can be in contact in at least one portion thereof with the counter-blade portion 24 integral in rotation with the reaction link 20, exchanging frictional forces directed substantially in the opening/closing direction G.

In accordance with a preferred embodiment, said counter-blade portion 24 integral in rotation with the reaction link 20 protrudes axially to bend the body of the blade link 30. The provision of such a counter-blade portion 24 integral in rotation with the reaction link 20 and protruding allows it to abut against the cutting edge 34 of the blade link 30, bending the body of the blade link 30. In accordance with an embodiment, the protrusion of the counter-blade portion 24 is accentuated in a distal direction along the longitudinal extension of the body of the reaction link 20.

In accordance with an embodiment, said counter-blade portion 24 integral in rotation with the reaction link 20 comprises a curved protruding surface having a concavity facing axially inwards. Thereby, the protrusion of the counter-blade portion 24 is given by the curvature thereof having a concavity facing axially inwards.

In accordance with an embodiment, the counter-blade portion 24 integral in rotation with the reaction link 20 protrudes towards the rotational footprint of the blade link 30, to elastically bend the body of the blade link 30 when the counter-blade portion 24 is in mechanical interference contact with the cutting edge 34. In other words, the counter-blade portion 24 protrudes axially inwards. In accordance with an embodiment, said protruding of the counter-blade portion 24 accentuates towards the distal direction, i.e., away from the common rotation axis Y-Y along the longitudinal extension of the reaction link 20 and preferably said protruding is maximum close to or at the distal end 32 of the body of the blade link 30.

Preferably, the term “approaching rotational footprint” is meant to indicate the volume of space which can be occupied by the body of an element during the relative rotation movement of the closing of the degree of freedom of grip G.

In accordance with an embodiment, a counter-blade link 40 is provided comprising in a single piece said counter-blade portion 24, in which the counter-blade link 40 is integral in rotation with the reaction link 20. Preferably, the counter-blade link 40 comprises in a single piece a proximal attachment root 41, said counter-blade portion 24 and a constrained distal end 42, and the reaction link 20 comprises in a single piece the root 21 and a distal free end, in which the root 41 of the counter-blade link 40 and the root 21 of the reaction link 20 are side by side and in direct and intimate contact with each other. Where said counter-blade link 40 is provided, then the group formed by said root 51 of the blade holder link 50, and said root 31 of the blade link 30, and said root 41 of the counter-blade link 40 and said root 21 of the reaction link 20 is globally interposed between said two prongs 3, 4 of the distal connection portion of the support link 2 and in direct and intimate contact therewith.

The counter-blade link 40 can be made by the sharpening and shaping steps, and in this case will comprise a cutting edge 34 and can be made from a workpiece 204.

In accordance with an embodiment, the counter-blade link 40 comprises a drag engagement portion 47 which engages with a drag engagement portion 67 of the reaction link to rotate the counter-blade link 40 and the reaction link 20 together.

Not necessarily the blade surface 35 is a flat portion, i.e., lying on a plane and can be a curved or arched portion, although in accordance with an embodiment it is a flat portion.

In accordance with an embodiment, the body of the blade link 30 has a two-dimensional main extension, i.e., lying on a preferably flat or arched lying surface, and has a substantially reduced thickness with respect to the extension on said preferably flat or arched lying surface.

In accordance with an embodiment, the cutting edge 34 is substantially straight in the preferably flat or arched lying surface, avoiding providing concavities in the lying surface of the body of the blade link 30.

Preferably, the thickness of the blade link 30 is significantly less with respect to the thickness of the attachment root 51 of the blade holder link 50 and the attachment root 21 of the reaction link 20, and the body of the blade link 30 is chosen so that it is elastically bendable when in operating conditions, transversely to the longitudinal extension of the cutting edge 34, and in particular in the direction of the thickness of the blade link 30. In particular, the body of the blade link 30 is preferably more bendable than the body of the reaction link 20, and preferably also more flexible than the body of the counter-blade portion 24. The flexibility of the blade link 30 and thus the flexibility of the cutting edge 34 is intended in the direction of the thickness thereof, i.e., in a direction orthogonal to the lying surface, whether flat or arched, of the blade link 30. For example, the blade link 30 has an arched, i.e., concave, conformation having a concavity facing in a direction exiting from/entering into the lying plane and in such a case the lying surface of the body of the blade link 30 is an arched surface as is the blade surface 35.

Not necessarily the body of the blade link 30 and thus the cutting edge 34 must be elastically deformable in the lying surface, i.e., a bendability in a direction orthogonal to the thickness thereof is not necessarily provided.

The ratio between the thickness of the body of the blade link 30 at the level of the blade portion 14 (excluding in this evaluation the thickness of the cutting edge 34, which as mentioned is preferably sharpened) and the thickness of the root 51 of the link 50 and/or the thickness of the second root 21 of the reaction link 20 can be between ⅕ and 1/20. In absolute value the thickness of the blade link 30 can be between 0.1 mm and 0.5 mm and in accordance with an embodiment substantially equal to 0.2 mm.

As mentioned above, the blade link 30 is integral in rotation with the blade holder link 50. Thereby, the cutting edge 34 is integral in rotation with a distal free end which can be formed by the body of the blade holder link 50 and/or the body of the blade link 30. In the case where the free end is formed by the body of the blade link 30 it can coincide with the distal end 32 of the blade link 30. Being elastically flexible, the cutting edge 34 can be elastically deformed with respect to the blade holder link 50 integral therewith in rotation when in operating conditions. The elastic deformation of the cutting edge 34 preferably occurs in a transverse direction with respect to the longitudinal extension direction of the body of the blade holder link 50, i.e., in a transverse direction with respect to the direction joining the proximal attachment root 51 and the free end integral in rotation to the cutting edge 34, in other words in the direction of the thickness of the body of the blade link 30.

In accordance with an embodiment, the blade link 30 is substantially planar when in a non-deformed configuration, i.e., it lies on a definable lying plane. The bending elasticity of the blade link 30 tends to bring the blade portion 14 back into said non-deformed planar configuration.

The cutting edge 34 can be aligned with the longitudinal extension direction X-X of the shaft 7 in at least one operating configuration, for example in the case in which the shaft 7 is a straight and rigid shaft and the cutting edge 34 is out of contact with a protruding portion of the counter-blade portion 24.

In accordance with an embodiment, said counter-blade portion 24 is a curved surface. Thereby, the counter-blade portion 24 protrudes due to the arched shape thereof. The concavity of the counter-blade portion 24 is preferably axially and facing inwards i.e., in a direction parallel to the common rotation axis Y-Y and facing the rotational footprint of the cutting edge 34.

The counter-blade portion 24 can act as a wedge to appropriately bend the cutting edge 34 and the body of the blade link 30 to exert the cutting action substantially along the entire longitudinal extension of the counter-blade portion 24.

As mentioned above, the support link 2 is made by wire electro-erosion starting from a workpiece 302 on which two shaping cuts are made on planes which are orthogonal to each other, and also the blade holder link 50 is made by wire electro-erosion starting from a workpiece 350 on which two shaping cuts are made on planes which are orthogonal to each other, and also the reaction link 20 is made by wire electro-erosion starting from a workpiece 320 on which two shaping cuts are made on planes which are orthogonal to each other. The connection link to the shaft, where present, can also be made by wire electro-erosion starting from a workpiece 390 on which two shaping cuts are made on planes orthogonal to each other. Instead, the blade link 30 is made by wire electro-erosion from a workpiece 204 on which two cuts are made on non-orthogonal planes where one cut is a sharpening cut.

In accordance with an embodiment, all the links of the articulated end-effector are made by wire electro-erosion and are assembled together.

In accordance with a general embodiment, a robotic surgery system 101 is provided, comprising at least one surgical instrument 1 according to any one of the embodiments described above. The robotic surgery system 101 is thus capable of performing surgical or microsurgical procedures including cutting a biological tissue and/or cutting sutures.

In accordance with an embodiment, said robotic surgery system 101 comprises at least two surgical instruments, at least one of which is a surgical instrument 1 according to any one of the embodiments described above and the other surgical instrument can be a surgical instrument of the needle-driver type or a surgical instrument of the dilator type, although in accordance with an embodiment both surgical instruments are surgical instruments 1 according to any one of the embodiments described above, not necessarily mutually identical although they can be. For example, a surgical instrument of the at least two surgical instruments can be a surgical instrument of the surgical scissor type and another surgical instrument of the at least two surgical instruments can be a surgical instrument of the needle-driver/scissor type.

The robotic surgery system 101 preferably comprises at least one robotic manipulator 103 and the at least one surgical instrument 1 is operatively connected to said at least one robotic manipulator 103. For example, a sterile surgical barrier (not shown), such as a sterile surgical cloth, for example, is interposed between the at least one robotic manipulator 103 and the backend portion 104 of the at least one surgical instrument 1. The robotic manipulator 103 can comprise motorized actuators for stressing said actuation tendons of the degrees of freedom of pitch P, yaw Y and grip G, i.e., cutting G of the surgical instrument 1, and a motorized actuator for rotating the surgical instrument 1 about the shaft 7 defining a degree of freedom of roll R. The robotic surgery system 101 can comprise a support portion 106 (cart or tower) for example comprising wheels or other ground contact units, and an articulated positioning arm 105, for example manually movable i.e., passive, extending between the support portion 106 and the at least one robotic manipulator 103. In accordance with an embodiment, the robotic surgery system 101 comprises at least one master console 107 for controlling the at least one surgical instrument 1 and preferably also the respective robotic manipulator 103 according to a master-slave architecture, and preferably the robotic surgery system 101 further comprises a control unit operatively connected to the master console 107 and the robotic manipulator 103 for determining the tracking of the surgical instrument 1 to at least one master control device 108 of the master console 107. In accordance with an embodiment, the master console 107 comprises at least one master control device 108 which is unconstrained, i.e., mechanically disconnected from the ground, and a tracking system, for example optical and/or magnetic.

In accordance with a general embodiment, the manufacturing method by electro-erosion according to any one of the previously described operating modes obtains one or more sharp bodies which are not necessarily intended to perform a cutting action when in operating conditions.

Furthermore, said sharp bodies made by the manufacturing method are not necessarily intended for applications in the medical field.

In accordance with an operating mode, said sharp bodies made by the manufacturing method are intended for use in one or more of the following technical domains: watchmaking, jewelry, costume jewelry, precision mechanics, electronics, nano-technology. The sharpening can have a cutting and/or aerodynamic and/or electrical and/or electro-magnetic and/or thermal and/or aesthetic function. For example, watch hands having a sharp edge can be made with such a method. For example, they can be made with such micro-antenna having sharp edges.

It is well understood that the combination of features, structures or functions disclosed in one or more of the appended claims forms an integral part of the present description.

By virtue of the features described above, provided either separately or in combination with one another in particular embodiments as well as operating modes, it is possible to meet the needs mentioned above, and to obtain the aforementioned advantages, and in particular:
it allows obtaining excellent surface finishes of the walls made by through cuts by wire electro-erosion (WEDM) and this favors a boosted miniaturization of the product of the manufacturing process;
at the same time, two non-orthogonal cuts are made for shaping and sharpening the same workpiece, avoiding repositioning the piece to be machined and thus further increasing the finish;
a “no-back-bevel” or “chisel edge” type sharpening is allowed with one or more passes of the cutting edge along a single sharpening cutting path;
it allows making a sharp elastic body, for example a blade;
it is possible to produce a plurality of sharp bodies with a single continuous cutting action from a single workpiece, for example a plurality of blades;
the rotation angle of the fixture from the sharpening step to the shaping step, or vice versa, is different from 90°;
where two shaping steps are provided, the rotation angle of the fixture from the shaping step to the reshaping step is again substantially 90°;
the shaping step can comprise the step of leaving bridges of material intact, making a semi-finished product 250;
the coating step can be performed on the semi-finished product 250 after performing the sharpening step and/or on the workpiece 204 before performing the sharpening step;
the shaping step can comprise the step of separating the sharp bodies from the workpiece.

In order to meet specific, contingent needs, those skilled in the art can make several changes and adaptations to the above-described embodiments and can replace elements with other functionally equivalent ones, without departing from the scope of the appended claims.

### LIST OF REFERENCE SIGNS

1 Surgical cutting instrument
2 Support link
3 Support link prong
4 Support link prong
9 Articulated end-effector of the surgical cutting instrument
15 Termination seat of the blade holder link
20 Reaction link
21 Reaction link root
24 Counter-blade portion
25 Termination seat of the reaction link
30 Sharp body, or blade, or blade link
31 Blade link root
32 Distal end
34 Sharp edge
36 Hole edge or hole rim
37 Blade link drag portion
39 Radial cutting channel
40 Counter-blade link
41 Counter-blade link root
42 Distal counter-blade link end
47 Drag portion of the counter-blade link
50 Blade holder link
51 Blade holder link root
57 Drag portion of the blade holder link
67 Drag portion of the reaction link
79 Convex ruled surface of the blade holder link pulley portion
80 Convex ruled surface of the reaction link pulley portion
96 Convex ruled surface of the support link
98 Convex ruled surface of the support link
200 Wire electro-erosion machine
202 Cutting wire
204 Workpiece
205 Workpiece flap or margin
206 Lower head of the electro-erosion machine
207 Upper head of the electro-erosion machine
208 Electro-erosion machine tank
209 Electro-erosion machine reel
210 Workpiece thickness
211 Conduit
212 Pump
213 Nozzle
214 Jig or fixture
215 Jig fixing portion
216 Electro-erosion machine bracket
217 First rotatable portion for housing the jig
218 Jig or fixture motor
219 Fixing element
220 Jig stroke end
221 Rectified jig surface
222 Rectified jig surface
223 Workpiece cutting wall
224 Workpiece back face
225 Workpiece front face
228 Surface treatment, e.g., coating and/or heat treatment
229 Calibration reference point
230 Shaping cutting trajectory or path
231 Connection bridge
232 Starting point of a shaping cutting trajectory or path pass
233 Ending point of a shaping cutting trajectory or path pass
234 Workpiece edge to be sharpened
235 Starting point of a sharpening cutting trajectory or path pass
236 Ending point of a sharpening cutting trajectory or path pass
238 External cutting profile section
239 Cutting profile notch
240 Sharpening cutting trajectory or path
241 Longitudinal slot of the jig housing portion
242 Control system
243 Bowl
250 Semi-finished product
260 Press
264 Press hammer
262 Press anvil
270 Second rotatable portion for housing the jig
302 Piece to be shaped
304 Second workpiece for sharpening
320 Piece to be shaped
350 Piece to be shaped
390 Piece to be shaped
F-F Jig rotation axis
X-X Longitudinal shaft direction
α Sharpening rotation angle
β Acute angle of the sharp edge
W Cutting wire feeding direction, or cutting direction

1. A method of manufacturing one or more sharp bodies by wire electro-erosion, comprising:
providing a wire electro-erosion machine comprising a cutting wire and a fixture which is rotatable with respect to the cutting wire about a rotation axis which is transverse to a longitudinal extension of the cutting wire;
mounting at least one workpiece on the fixture;
sharpening at least one edge to be sharpened of the at least one workpiece by performing a sharpening through cut with the cutting wire on the at least one workpiece;
shaping the at least one workpiece by performing a shaping through cut with the cutting wire on the at least one workpiece;
wherein between the sharpening and the shaping a further step is performed of:
rotating the fixture about the rotation axis by a sharpening rotation angle other than 90°.

2. The method according to claim 1, wherein said one or more sharp bodies comprise one or more surgical blades and the method is a method of manufacturing one or more surgical blades by wire electro-erosion.

3. The method according to claim 1, wherein the method makes a plurality of sharp bodies on a single workpiece, and
wherein said sharpening and said shaping are the same for all the sharp bodies of said plurality.

4. The method according to claim 1, wherein the sharpening step is performed with a single cutting sharpening trajectory of the cutting wire and said shaping step is performed with a single cutting shaping trajectory of the cutting wire.

5. The method according to claim 1, wherein the sharpening rotation angle is an acute angle.

6. The method according to claim 1, wherein the sharpening is performed first, followed by the rotating, then the shaping.

7. The method according to claim 6, wherein the shaping through cut of the shaping crosses at least one portion of the sharp edge.

8. The method according to claim 6, wherein the shaping comprises separating the sharp bodies due to the shaping through cut; and wherein the method further comprises collecting the separate sharp bodies in a collection basket by gravity.

9. The method according to claim 1, wherein the at least one workpiece comprises a plate-like body having a thickness, and the sharpening and shaping steps each provide making a through cut through the thickness of the plate-like body of the at least one workpiece.

10. The method according to claim 1, wherein said workpiece is a plate-like body having a thickness in the range 0.05 mm-0.5 mm, wherein the at least one workpiece comprises an elastic body which is elastically deformable by bending.

11. The method according to claim 1, wherein the sharp edge of the sharp body is a curved edge.

12. The method according to claim 1, wherein the shaping step comprises shaping at least one hole edge on the workpiece, said hole edge being configured to delimit a through hole through a thickness of the sharp body.

13. The method according to claim 1, further comprising after the shaping:
reshaping the workpiece on a second cutting plane, performing a second shaping through cut with the cutting wire on the at least one workpiece;
wherein between the shaping and the reshaping:
rotating said fixture by a shaping angle preferably substantially equal to 90°, and wherein the sharpening is performed before the shaping and/or wherein said sharpening is performed between said shaping step and said reshaping.

14. The method according to claim 1, wherein the mounting comprises mounting a plurality of workpieces on said fixture; and wherein the sharpening and shaping individually comprise sharpening and shaping each workpiece of said plurality of workpieces.

15. The method according to claim 1, wherein the mounting comprises mounting at least a second workpiece on said fixture to obtain at least two workpieces mounted on the same fixture; and wherein the method further comprises:
sharpening at least one edge to be sharpened of said second workpiece; and wherein between sharpening at least one edge to be sharpened of the at least a first workpiece and sharpening at least one edge to be sharpened of said second workpiece, of rotating at least one portion of said fixture.

16. The method according to claim 1, further comprising zeroing and calibrating the electro-erosion machine comprising:
identifying a reference point for a cutting path or trajectory,
approaching said reference point with the cutting wire before the sharpening;
wherein said reference point belongs to the edge to be sharpened of the workpiece.

17. The method according to claim 1, wherein the identification comprises identifying a single starting point which acts as a point of origin for both the sharpening cutting path and the shaping cutting path; and wherein the approaching comprises approaching said single point of origin with the cutting wire both in preparation for the sharpening and in preparation for the shaping; and
wherein the single starting point is in a predefined geometric relationship with the reference point.

18. The method according to claim 16, wherein between the identification and the sharpening and/or shaping a rotation is performed along said rotation axis by an acute angle.

19. The method according to claim 1, wherein the sharpening through cut is performed with repeated multiple passes of the cutting wire along a same sharpening cutting path, wherein the number of said repeated plurality of passes of the cutting wire for performing said sharpening through cut is greater than the number of passes made for performing the shaping through cut.

20. A semi-finished product comprising a plate-like body in a single piece having a plurality of sharp bodies, shaped and connected together by one or more connecting bridges, wherein the plate-like body of the semi-finished product comprises an edge comprising a plurality of sharp edges.

21. A fixture for an electro-erosion machine comprising a fixing portion for mounting the fixture to the electro-erosion machine and a housing portion for receiving at least one workpiece, wherein the housing portion is rotatable with respect to the fixing portion about a rotation axis.

22. A fixture according to claim 21, comprising a motor for rotating the housing portion with respect to the fixing portion.

23. A fixture according to claim 21, comprising a plurality of housing seats for receiving a plurality of workpieces, wherein said housing seats are arranged so as not to overlap in two mutually incident and coplanar directions.

24. The method according to claim 1, wherein the sharpening rotation angle is in the range 20°-70°.

25. The method according to claim 1, wherein the sharpening rotation angle is in the range 30°-60°.

26. The method according to claim 1, wherein the sharp edge of the sharp body is concave and/or convex in a lying plane of the sharp body.
